(19) United States
(12) Patent Application Publication
(10) Pub. No.: US 2016/0310632 A1
(43) Pub. Date: Oct. 27, 2016

(54) IMPROVED TWO-SOLUTION BONE CEMENT

(71) Applicant: BOARD OF REGENTS, THE UNIVERSITY OF TEXAS SYSTEM, Austin, TX (US)

(72) Inventors: Danieli C. RODRIGUES, Richardson, TX (US); Lucas C. RODRIGUEZ, Dallas, TX (US); Jonathan CHARI, Dallas, TX (US)

(21) Appl. No.: 15/105,703

(22) PCT Filed: Dec. 8, 2014
(86) PCT No.: PCT/US2014/069032

§ 371 Date: Jun. 17, 2016

Related U.S. Application Data
(60) Provisional application No. 61/919,173, filed on Dec. 20, 2013.

(51) Int. Cl. A61L 24/00; A61L 24/02; A61L 24/04

(52) U.S. Cl. CPC A61L 24/0015; A61L 24/001; A61L 24/046; A61L 24/02; A61L 24/0036; A61L 2430/02

(57) ABSTRACT

The present disclosure relates to the treatment of bone disease and injuries using improved bone cements and to stabilize and restore bone structures in vivo. The bone cements employ a linear polymer; a monomer; a bioactive mineral-containing agent; and an antimicrobial agent.

The present application claims benefit of priority to U.S. Provisional Application Ser. No. 61/919,173, filed Dec. 20, 2013, the entire contents of which are hereby incorporated by reference.

### BACKGROUND

1. Field

The field of the disclosure is medicine, and more specifically to the treatment of bone disease and injuries using artificial materials. In particular, the field relates to the use of improved bone cements and their use to stabilize and restore bone structures in vivo.

2. Background

Acrylic bone cements are utilized in orthopedic applications ranging from trauma situations to elderly patients with diminished bone quality. New bone cement formulations have incorporated calcium phosphate fillers to induce osteoconductivity and improve osteointegration, which could result in better performance for the patient receiving a cemented implantation or augmenting fractured bone. Addition of calcium phosphate (CaP) fillers on the other hand, has typically resulted in detrimental effects in both experimental and commercial formulations. These fillers have been reported to cause increased cement viscosity, decreased handling, and detrimental effects of the mechanical performance of the cement (Ishiguro et al., 2010; Ryu et al., 2010; Blattert et al., 2009; Wilke et al., 2006; Weiss et al., 2003). Bohner et al. (Bohner & Baroud, 2005) demonstrated that as the powder-to-liquid (P:L) ratio of the cement composition increased (in powder-liquid cement compositions), the injectability of the cement was detrimentally affected. Further, many CaP cements are not suitable for load bearing applications because their compressive strengths are not to the standard of acrylic bone cements used for implant augmentation [7] (Grover et al., 2003).

Because bacterial infection remains a serious complication following trauma and orthopedic implant surgery, bone cements have also been offered with antibiotics/antimicrobials in the formulation. Although infection rates after initial augmentation surgery are currently around 1-2%, these cases are responsible for multiple operations, amputations, and sometimes mortality for the patient, as well as lawsuits for the hospital and physician performing the operations (Hendriks et al., 2004). Thus, the induction of antimicrobial activity in bone cements against various microorganisms, which are commonly found in osseous cavities, provides the cement formulations with the ability to combat the bacteria at the primary site. Typically bone cements are loaded with gentamicin, vancomycin, trombamycin, several cephalosporins, with gentamicin being the most widely used. Previous studies have demonstrated that these antimicrobials are capable of eluting out of the polymerized cement matrix at concentrations well above the minimal inhibitory concentration (MIC) (Rosenthal et al., 1976). These antimicrobial loaded bone cements have significantly improved efficacy in preventing post-operative infections as compared to non-loaded cements (Josefsson et al., 1981). However, results have also illustrated the detrimental effect that antimicrobial fillers have on material fatigue life, compressive strength, and tensile strength (Klekamp et al., 1999; Lautenschlager et al., 1976). Gentamicin has been broadly used in bone cements because of its wide spectrum and well-established concentration-dependent activity (Lacy et al., 1998). There are certainly problems associated with the addition of antimicrobials to bone cements. A well-documented side effect of over-exposure to gentamicin is kidney damage (Trippel, 1986). An unstoppable/consistent dosage of gentamicin could cause further damage as gentamicin concentrations increase over time due to cement being replaced with natural bone (as the case of CaP-containing cement compositions). Further, because the antimicrobials are entrapped within the cement, there is an ongoing/continuous release of the substances over long periods of time. However, because the acrylic nature of the cement renders it mostly non-resorbable, up to 80% of the antimicrobial agent may be permanently “locked” within the bone cement matrix (Wroblewski et al., 1986; Powles et al., 1988). Resistance to such components becomes a concern due to the prolonged exposure (Hendriks et al., 2004; Van de Belt et al., 1999). Therefore, there is an emerging interest in alternative and effective antimicrobials for addition in bone cements.

### SUMMARY

Thus, in accordance with the present disclosure, there is provided an orthopedic bone cement comprising:
(a) a particles;
(b) a linear poly (methyl methacrylate) polymer;
(c) a methyl methacrylate monomer;
(d) a bioactive calcium phosphate-containing agent;
(e) a porosity agent;
(f) an initiator of polymerization; and
(h) an activator of the initiator,
(h) an inhibitor of polymerization.
   The total polymer to monomer ratio may be between about 1:1 and 3:1, or between about 1:1 and 2:1, or between about 1:1 and 1.5:1, or between about 1:1 and 1:2, or between about 1:1 and 1:3, and in particular 1:3. The poly(methyl methacrylate) may be about 60,000 to about 1000,000 daltons. The particles may comprise microspheres. The particles may be between about 0.1 and 400 micrometers, or between about 1.0 and about 100 micrometers. The powder-to-liquid ratio may be about 1:1 to 2:1, or about 1:1 to 1.5:1, and in particular about 1.31:1.

The cement may further comprise an antimicrobial agent such as antibiotic, such as chlorhexidine, vancomycin, tobramycin or gentamicin, or an antifungal. The antimicrobial agent maybe present at 1-10 weight percent, including 1-8 weight percent and 1-6 weight percent. The bioactive calcium phosphate-containing agent may be tricalcium phosphate, brushite or hydroxyapatite. The bioactive calcium phosphate-containing agent may be included at 25-50% wt/wt of PMMA, at 25% wt/wt of PMMA, at 30% wt/wt of PMMA, at 40% wt/wt of PMMA or at 50% wt/wt of PMMA.

The monomer may comprise of a mixture of methyl methacrylate monomer (MMA) and styrene. The porosity agent may be lactose. The initiator of polymerization is may be benzoyl peroxide. The activator of the initiator may be electromagnetic radiation with wavelength between 10⁻⁷ to 10⁻¹⁰ meters. The inhibitor of polymerization may be hydroquinone. The activator of the initiator may be N,N-Dimethyl-Para-Toluidine.

Also provided is a method of treating a subject for a bone-related disorder comprising administering to a site of bone damage, loss or deficiency a bone cement according to the description immediately above. The method may further comprise implanting into said subject medical device or appliance. The term treating may comprise an orthopedic, periodontal, neurosurgical, oral or maxillofacial procedure. The term treating may comprise repair of a simple fracture, compound fracture or non-union; external or internal fixation; joint reconstruction, arthrodesis, arthroplasty or cup arthroplasty of the hip; femoral or humeral head replacement; femoral head surface replacement or total joint replacement; repair of the vertebral column, spinal fusion or internal vertebral fixation; tumor surgery; deficit filling; discectomy; laminectomy; excision of spinal cord tumors; an anterior cervical or thoracic operation; repair of a spinal injury; treatment of scoliosis, treatment of lordosis; kyphosis treatment; intermaxillary fixation of a fracture; mentoplasty; temporomandibular joint replacement; alveolar ridge augmentation or reconstruction; as part of an inlay osteoimplant; implant placement and revision; sinus lift; a cosmetic procedure; or the repair or replacement of the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal bones and/or metatarsal bones.

Whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and are not limiting. For example, the term “including” shall mean “including, but not limited to.”

The term “about” is intended to convey a deviation from a stated value or range of +/−5%.

It is contemplated that any embodiment discussed in this specification can be implemented with respect to any compound, method, or composition, and vice versa.

Other objects, features and advantages will become apparent from the following detailed description. It should be understood, however, that the detailed description and the specific examples, while indicating specific embodiments, are given by way of illustration only, since various changes and modifications within the spirit and scope will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. The material's compressive strength was determined by acquiring the upper yield point of the material and the 2% offset yield. The value of whichever occurred first was used as the materials compressive strength as indicated by ASTM FO451 (ASTM Standard F451, 2008).

FIGS. 2A-B. Multiple comparison evaluation of cement compositions tested. (FIG. 2A). The compressive strength of reagent grade chlorhexidine samples were all significantly lower than the control cement groups evaluated (p<0.05). As the concentration of chlorhexidine within the samples was increased, the compressive strength decreased. The 80% increase in initiation chemistry was able to recover compressive strength up to an average of only 49%. (FIG. 2B) The chlorhexidine diacetate samples were all significantly similar (p>0.05) to the control samples evaluated. All samples tested with chlorhexidine diacetate retained their mechanical properties.

FIGS. 3A-C. Bacterial proliferation after cement exposure. (FIG. 3A) Control samples without antimicrobials and samples evaluated with chlorhexidine concentrations below 8% all eventually proliferated to above 0.2 critical optical density. All samples containing 8% chlorhexidine (FIG. 3B) and/or 8% chlorhexidine diacetate (FIG. 3C) displayed an ability to resist antimicrobial proliferation. Each of these select samples remained well below the critical optical density of 0.2.

FIG. 4. Proposed mechanism of propagating chain interaction with chlorhexidine diacetate. The ionic interaction between the acetate ion and the chlorhexidine molecule shields it from attack from the free radical.

FIG. 5. Determination of the LVR using oscillation strain sweep. The region to the left of the indicated line represents the Linear Viscoelastic Region, where the stress and strain magnitudes are linearly related. Thus a material's behavior is able to be modeled independent of the magnitude of applied strain. Outside of the LVR, this no longer remains true.

FIG. 6. Acquisition of extrusion stress using the stress recorded at near-zero shear.

FIG. 7. Complex viscosity of several non-setting cement compositions after the incorporation of calcium phosphate additives at varying oscillation frequencies. Non-setting cements were evaluated in order to extend the working phase of the cement, which enabled understanding of the material's behavior during the doughy state (label on left top to bottom is the same as bar graphs left to right).

FIG. 8. Average complex viscosity of non-setting calcium phosphate TSBC's investigated at varying angular frequencies to illustrate the shear thinning of the material irrespective of angular frequency or composition. The multiple comparison graph illustrates the similarity of the values at a 95% confidence interval. Samples from top to bottom in graph: 50% TCP, 50% Brushite, TSBC, 25% HA, 25% TCP, 25% Brushite, 50% HA.

FIG. 9. 200× digital imaging of TSBC cement compositions to investigate topography of the cement surfaces.

FIG. 10. Determination of optimum cement composition. The optimum composition of the investigative material is that which allows for the maximum amount of CaP filler (Brushite), without changing the rheology of the material within the different aspects of the cartridge. Testing suggests that the CBS E2 material demonstrates the optimum viscosity throughout the cartridge for the applications referenced. This material has a P:L (powder-to-liquid) ratio of 1.31:1. The material's powder component is 68% CaP (Brushite) and 30% PMMA allowing for a disperse scaffold to develop as the CaP is resorbed.

### DETAILED DESCRIPTION

The inventor has investigated the feasibility and effectiveness of a novel bioactive two-solution bone cement, meaning that the material integrates with bone. The present disclosure discusses the development and characterization of the two-solution cement with the incorporation of a mineral phase such as HA and brushite as examples employing CaP, and an antimicrobial agent. Chlorhexidine (CHX) was investigated as an example of an antimicrobial agent in these cement compositions due to its wide spectrum as well as its vast use in dentistry. Although chlorhexidine has not been investigated widely in acrylic bone cements, it has been examined in dental cements. For example, a few studies have attempted incorporating CHX in Mineral Trioxide Aggregate (MTA), which is a commonly utilized root-end filling material. While CHX is a very attractive antimicrobial for such application, one shortcoming of the MTA material in dental cements is its inability to incorporate chlorhexidine into its matrix while retaining the cement compressive strength (Kogan et al., 2006). However, because the antimicrobial effectiveness of chlorhexidine-containing cements is undisputed, its addition to bone cement will provide antimicrobial activity to the cement (Holt et al., 2007; Stowe et al., 2004).

The bioactive and antimicrobial two-solution cements discussed in this disclosure were designed to have adjustable properties that meet specific requirements of orthopedic applications, such as arthroplasty, fracture augmentation, and bone void fillers. The addition of a bioactive agent would lead to increased levels of bone reformation after surgery, while an antibiotic within the cement would decrease the ability for pathogens to grow in the interface between the bone and new implant. However, the compressive strength of the cement in these applications is pivotal, and it was thus crucial to demonstrate that the addition of antimicrobials and mineral (especially CaP) fillers do not detrimentally affect the mechanical properties of the cement.

As shown below, this is precisely what has been demonstrated. In particular, the inventor has shown that two-solution bone cements are capable of incorporating calcium phosphate and an antimicrobial agent without negatively affecting the mechanical properties of the material. By controlling the free radical quenching mechanism caused by the chlorhexidine molecule, it was possible to achieve high polymer conversion rates. This phenomenon led to strength retention while successfully preventing microbial proliferation in an environment exposed to the cement surface. Thus, the results of these initial studies are remarkable in that they indicate the addition of chlorhexidine as antimicrobial agent in cements used in load bearing applications may now be a viable option. The applications of cements with bone mimicking characteristics are many, ranging from a trauma patient needing an augmentation of a fracture, to a cancer patient in need for a bone void filler after a tumor excision.

These and other aspects of the disclosure are discussed below.

### I. TWO-SOLUTION BONE CEMENT

Standard two-solution bone cement (described in U.S. Pat. No. 5,902,839), and here referred as TSBC, has emerged as an alternative to current powder-liquid formulations. The standard two-solution cement (TSBC) has the advantage of being porosity free and having a higher flexural strength and modulus of elasticity than powder-liquid formulations. It also has the advantage of being pre-mixed, which allows the cement to be mixed at one time, left to homogenize and used at a later time. This ensures that additives have time to disperse through their respective solutions. Furthermore, this allows the metered delivery of the cement in a closed system, which minimizes the risk of contamination and exposure to operating room personnel. (see Hasenwinkel et al., 1999; 2002). However, one limitation of the original TSBC formulation was the higher initial viscosity for a relatively low polymer-to-monomer ratio (P:M of 0.9:1), which resulted in increased residual monomer concentration, and short setting time. Because the viscosity of the original two-solution cement (TSBC) was high at a relatively low P:M ratio, its application was very limited to using delivery devices that applied high pressures. To address this issue, the composition of the cement was manipulated by incorporating cross-linked PMMA microspheres (μ-TSBC) or nanospheres (η-TSBC) (described in U.S. Pat. No. 5,902,839), and by the addition of polymer brushes (Rodrigues et al., 2009). The addition of cross-linked PMMA particles and replacement of the polymer phase by PMMA brushes allowed for an increase in the P:M ratio of the cement from 0.9:1 to up to approximately 1.1:1 without further increasing the viscosity of the cement. In fact, these cements exhibited improved handling because of the increased shear-thinning (pseudoplastic) nature achieved with cross-linked phase incorporation. The addition of cross-linked particles and brushes, although effective in controlling viscosity, demonstrated to be a time consuming process by requiring the synthesis of new phase for addition in the material. Most importantly, the TSBC containing cross-linked PMMA particles still lacked the ability to integrate with bone.

The new two-solution system described in this disclosure is an improvement over these previous cements and combines such properties such as bioactivity, high strength, modifiable viscosity, high pseudoplasticity, and ability to incorporate an effective antimicrobial agent and could serve the purpose of many applications. This new two-solution cement is intended to provide surgeons and patients with a material that is ready to use without the need of further modifications during surgery. The present disclosure describes the development of such bone cement by the addition of various minerals and antimicrobial phases.

It is well known that acrylic bone cements are non-Newtonian or pseudoplastic fluids that undergo shear-thinning with increasing shear rates, presenting significant differences in the flow behavior depending on the chemical composition and how the cement is manipulated mechanically. The clinical significance of highly pseudoplastic cements is related to the fact that the material can be subjected to rapid thinning, which consequently enhances flow through a delivery system and into the interstices of the bone. Another important factor affecting viscosity of bone cements is the incorporation of polymer particles or fillers in the cement matrix. Polymer particle size and distribution (polydispersity), volume fraction and particle-particle interaction are factors that determine the rheological behavior of dispersed systems. Even though the effects of the size and size distribution of PMMA particles on the properties of acrylic bone cements are discussed in the literature, most of these studies involved the application of commercial samples of linear PMMA used in powder-liquid formulations. For example, Pascual et al. showed that the use of PMMA particles with average diameter in the 50-60 μm range and with wide size distribution significantly changed the maximum polymerization exotherm and setting characteristics of cement formulations (Pascual et al., 1996). Likewise, Hernandez et al discussed the influence of powder size distribution on the properties of cements used in KP and VP showing that cements with a high proportion of large PMMA beads (˜118 μm) to small beads (˜70 μm) presented suitable viscosity behavior and injectability (see Hernandez et al., 2006).

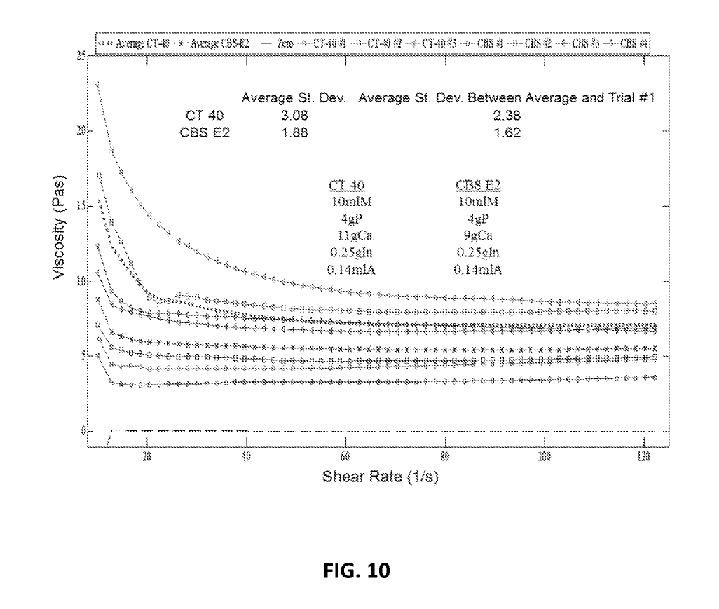

To overcome the lack of bioactivity of the cement, CaP fillers including Brushite (CaHPO₄.2_{H}2O), and Hydroxyapatite (Ca₅(PO4)₃(OH)) (HA) have been incorporated here into the TSBC in concentrations of 25-50% (wt/wt PMMA), and up to 50% (wt/mass) (FIG. 10), as examples of minerals that can be added. The rheological characteristics of these cements (Brushite-TSBC and HA-TSBC, respectively) were examined to verify the feasibility of adding CaP fillers to this cement formulation. Results demonstrated that unlike commercial powder-liquid formulations, these two-solution composite bone cements did not detrimentally affect handling or the rheological properties of the material. In addition to rheological testing, the mechanical testing of the two-solution composite bone cements demonstrated that the strength of TSBC is not detrimentally affected by the addition of CaP. In addition to bioactivity, TSBC has been given prophylactic properties here by the addition of an antimicrobial agent. Identical to the studies for the incorporation of CaP, the inventor has demonstrated that the addition of an antimicrobial agent did not detrimentally affect the mechanical, rheological, or handling properties of TSBC. The inventor has also demonstrated that the new HA-TSBC and Brushite-TSBC elutes the antimicrobial agent out of the polymerized cement matrix at concentrations well above the minimal inhibitory concentration (MIC).

U.S. Pat. No. 8,383,734 and U.S. Patent Publication 2009/0239970, describing TSBC's and methods of making and using the same, are hereby incorporated by reference.

A. Linear Polymers

Pre-polymerized polymers are added in the powder phase of typical bone cement formulations. A variety of chemicals may be employed in the preparation of polymers for addition in bone cements, in accordance with the present disclosure, including poly(methyl methacrylate) (PMMA), poly(hydroxyethyl methacrylate) (pHEMA), other polymerized methacrylates, poly(butyl acrylate), other polymerized acrylates, polystyrene, polyethylene, polyvinyl acetate, polyvinyl alcohol, poly(vinyl chloride), poly(vinylidene chloride), polybutadiene, poly(acrylic acid), alginic acid, acrylonitrile, β-linked D-glucosamine, N-acetyl-D-glucosamine, any derivative of glucosamine, or any combination of afore mentioned entities. The addition of linear polymers of high molecular weight will provide viscosity to the cement dough and result in high mechanical strength. Traditionally, acrylate based linear polymers are employed in the preparation of bone cements because of its well-known biocompatibility, performance, and long clinical history. In essence, the linear polymer is basically a low toxicity material of high molecular weight (>50,000 g/mol) to give mechanical strength, and miscibility with the remaining cement components.

B. Monomers

A variety of different monomers may be employed in accordance with the present disclosure, including methyl methacrylate (MMA), hydroxyethyl methacrylate (HEMA), other methacrylates, butyl acrylate, other acrylates, styrene, ethylene, vinyl acetate, vinyl chloride, vinylidene chloride, butadiene, acrylic acid, alginic acid, acrylonitrile, β-linked D-glucosamine, N-acetyl-D-glucosamine, or any combination of afore mentioned entities. Traditionally MMA, HEMA, other methacrylate derivatives have been employed as the monomer (or liquid) phase of bone cements because of the well-known biocompatibility and long clinical history.

C. Particles

A variety of different particles may be employed in accordance with the present disclosure, including linear and cross-linked PMMA particles, calcium phosphate, zirconium dioxide, barium sulfate, glass-reinforcing particles, lactose. While there are not set characteristics on the size of particles, typically it has been observed that the smaller the particle size, the higher the viscosity of the resulting cements dough. This is because the packing of smaller particles will result in agglomerates or clumps that are difficult to inject through small delivery devices.

D. Initiators of Polymerization

A variety of different polymerization initiators may be employed in accordance with the present disclosure, including benzoyl peroxide, dilauroyl peroxide, other peroxides and peroxy acids, tributylborane, camphorquinone, other chemical species capable of generating free radicals or any combination of aforementioned entities.

E. Activators of the Initiator of Polymerization

A variety of different initiation activators, both chemical and physical, may be employed in accordance with the present disclosure, including N,N-Dimethyl-Para-Toluidine, N,N-Dimethyl-Meta-Toluidine, any or any source of halides, any source of electromagnetic radiation or any combination of aforementioned entities.

F. Inhibitors of Polymerization

A variety of polymerization inhibitors may be employed in accordance with the present disclosure, including hydroquinone, any form or derivative of ascorbic acid, any tocopherol or its derivatives, any tocotrienol or its derivatives, any phenol or its derivatives, any thiol or its derivatives, any chemical entity capable of reacting with a free radical, or any combination of afore mentioned entities.

G. Porosity Agent

In order to maximize the release of antibiotics in a bone cement matrix or biodegradable material, a release modulator, such as lactose or hydroxypropylmethylcellulose, can be added to the materials. Lactose is a hydrophilic additive; therefore it will disintegrate in a polymer matrix leaving pores on the surface that can be used for scaffolding of new bone. The voids formed in the polymer matrix will aid in antibiotic release. Lactose can be added to acrylic PMMA containing bone cements that are loaded with antibiotics to modulate release, or in fully resorbable cements.

### II. BIOACTIVE CaP ADDITIVES

The properties of materials with CaP additives have been reported to cause the material properties to suffer (Ishiguro et al., 2010; Ryu et al., 2010; Blattert et al., 2009; Wilke et al., 2006; Weiss et al., 2003). Two-solution bone cements have been demonstrated in this application to better incorporate fillers into their matrix, without detrimentally affecting the material's properties. The ability of TSBC to incorporate CaP additives could further improve its in vivo performance.

The present disclosure will utilize bioactive CaP additives to improve the properties of previously described two-solution bone cements. These agents provide beneficial properties for orthopedic applications, including superior stabilization of compressed osteoporotic or diseased vertebrae, providing venues for biological interlocking, creating a scaffold for bone growth and integration with the remaining cement matrix, and being adequate for augmentation of trauma-related fractures in a younger patient population. As discussed in greater detail below, rheological evaluations have been conducted and no detrimental effects from the incorporation of CaP fillers into the investigated materials were observed. While not wishing to be bound by any theory, this is believed to be due to the increased polymer swelling time in the investigated materials as compared to commercially available powder-liquid formulations.

A. Hydroxyapatite

Hydroxyapatite (HA), also called hydroxylapatite, is a naturally occurring mineral form of calcium apatite with the formula Ca₅(PO₄)₃(OH), but is usually written Ca₁₀(PO₄)₆(OH)₂ to denote that the crystal unit cell comprises two entities. Hydroxyapatite is the hydroxyl end member of the complex apatite group. The OH⁻ ion can be replaced by fluoride, chloride or carbonate, producing fluorapatite or chlorapatite. It crystallizes in the hexagonal crystal system. Pure hydroxyapatite powder is white. Naturally occurring apatites can, however, also have brown, yellow, or green colorations, comparable to the discolorations of dental fluorosis.

Up to 50% of bone by weight is a modified form of hydroxyapatite (known as bone mineral). Carbonated calcium-deficient hydroxyapatite is the main mineral of which dental enamel and dentin are composed. Hydroxyapatite crystals are also found in the small calcifications (within the pineal gland and other structures) known as corpora arenacea or ‘brain sand’.

Hydroxyapatite can be found in teeth and bones within the human body. Thus, it is commonly used as a filler to replace amputated bone or as a coating to promote bone ingrowth into prosthetic implants. Although many other phases exist with similar or even identical chemical makeup, the body responds much differently to them. Coral skeletons can be transformed into hydroxylapatite by high temperatures; their porous structure allows relatively rapid ingrowth at the expense of initial mechanical strength. The high temperature also burns away any organic molecules such as proteins, preventing an immune response and rejection.

Many modern implants, e.g., hip replacements and dental implants, are coated with hydroxylapatite. It has been suggested that this may promote osseointegration. Porous hydroxylapatite implants are used for local drug delivery in bone.

B. Brushite

Brushite is a phosphate mineral with the chemical formula CaHPO₄.2H₂O. It forms colorless to pale yellow monoclinic prismatic crystals and as powdery or earthy masses. It is the phosphate analogue of the arsenate pharmacolite and the sulfate gypsum. Brushite is the original precipitating material in calcium phosphate kidney stones.

Brushite was first described in 1865, and is believed to be a precursor of apatite and is found in guano-rich caves, formed by the interaction of guano with calcite and clay at a low pH. It occurs in phosphorite deposits and forms encrustations on old bones. It may result from runoff of fields which have received heavy fertilizer applications. Associated minerals include tanarakite, ardealite, hydroxyapatite, variscite and gypsum.

C. Tricalcium Phosphate

Tricalcium phosphate (abbreviated TCP) is a calcium salt of phosphoric acid with the chemical formula Ca₃(PO₄)₂. It is also known as tribasic calcium phosphate and bone phosphate of lime (BPL). Calcium phosphate is one of the main combustion products of bone (see bone ash). Calcium phosphate is also commonly derived from inorganic sources such as mineral rock. It has an alpha and a beta crystal form, the alpha state being formed at high temperatures. As rock, it is found in Whitlockite.

Calcium phosphate refers to minerals containing calcium ions (Ca²⁺) together with orthophosphates (PO₄³⁻), metaphosphates or pyrophosphates (P₂O₇⁴⁻) and occasionally hydrogen or hydroxide ions. Especially, the common mineral apatite has formula Ca₅(PO₄)₃X, where X is F, Cl, OH, or a mixture; it is hydroxyapatite if the extra ion is mainly hydroxide. Much of the “tricalcium phosphate” on the market is actually powdered hydroxyapatite. Tricalcium phosphate occurs naturally in several forms, including as a rock (this form is not completely pure, and there are some other components like sand and lime which can change the composition), in the skeletons and teeth of vertebrate animals, and in in cow's milk.

Calcium phosphate is an important raw material for the production of phosphoric acid and fertilizers, for example in the Odda process. Phosphate ore quality and quantity is often specified as percent BPL (bone phosphate of lime), where 1% BPL is equivalent to 0.458% P₂O₅. Calcium phosphate is also a raising agent (food additive). As a mineral salt found in rocks and bones, it is used in cheese products.

It is also used as a nutritional supplement, although the most common and economical forms for supplementation are calcium carbonate (which should be taken with food) and calcium citrate (which can be taken without food). There is some debate about the different bioavailabilities of the different calcium salts. It is commonly used in porcelain and dental powders, and medically as an antacid or calcium supplement, although calcium carbonate is more common in this regard.

It can be used as a tissue replacement for repairing bony defects when autogenous bone graft is not feasible or possible. It may be used alone or in combination with a biodegradable, resorbable polymer such as polyglycolic acid. It may also be combined with autologous materials for a bone graft. Porous beta-tricalcium phosphate scaffolds are employed as drug carrier systems for local drug delivery in bone.

Another practical application of the compound is its use in gene transfection. The calcium ions can make a cell competent to allow exogenous genes to enter the cell by diffusion. A heat shock afterwards then invokes the cell to repair itself. This is a quick and easy method for transfection, albeit a rather inefficient one.

D. Other CaP Materials

Another material that can be employed in accordance with the present disclosure is strontium-hydroxyapaptite.

### III. RADIOPACIFIERS

A variety of radiopacifiers may be employed in accordance with the present disclosure, including barium sulfate, zirconium dioxide, calcium carbonate, calcium sulfate, strontium, or any combination of afore mentioned entities. These compounds are used to provide contrast to cements that require visualization of the flow during delivery in the body. Therefore, such agents are broadly used in dental cements and cements used for injection in the spine.

### IV. ANTIBIOTICS/ANTIMICROBIALS

As discussed above, while it is desirable to have antimicrobial activity in bone cements for use in orthopedic embodiments, in previous attempts to add antimicrobial agents there have been a number of problems relating to its implementation including, at one extreme, the failure of the agent to be readily transferrable into surrounding tissues, and at another, the toxicity from long-term exposure. However, the present disclosure contemplates the inclusion of various antibiotic and antimicrobial agents while at the same time not compromising the strength of the cement when applied in vivo.

A. Chlorhexidine

Chlorhexidine (reagent grade, diacetate, digluconate) is an antiseptic, first discovered and developed by Imperial Chemical Industries (ICI) and introduced under the brand name Hibitane. ICI also discovered and developed another antiseptic cetrimide and introduced Savlon which was a combination of both cetrimide and chlorhexidine. Chlorhexidine is effective on both Gram-positive and Gram-negative bacteria, although it is less effective with some Gram-negative bacteria. It has both bactericidal and bacteriostatic properties, the mechanism of action being membrane disruption, not ATPase inactivation as previously thought. It is also useful against fungi and enveloped viruses, though this has not been extensively investigated.

Chlorhexidine is often used as an active ingredient in mouthwash designed to reduce dental plaque and oral bacteria. It has been shown to have an immediate bactericidal action and a prolonged bacteriostatic action due to adsorption onto the pellicle-coated enamel surface. If it is not deactivated, chlorhexidine lasts longer in the mouth than other mouthwashes and this is partly why it is to be preferred over other treatments for gingivitis. To treat periodontal pockets equal or greater 5 mm chlorhexidine is also available in high concentration (36%) in a gelatine-chip (PerioChip).

There are oral pathologic conditions in which the maintenance of oral hygiene with the twice-daily use with 0.12% chlorhexidine-gluconate solution (in which a salt of chlorhexidine and gluconic acid has been dissolved) is required for healing and regeneration of the oral tissues. These conditions included gingivitis, periodontitis, dental traumas (such as subluxation), oral cysts, and after wisdom tooth extraction. The clinical efficacy of the application of chlorhexidine as a component of oral rinses is well documented by many clinical studies that are summarized by review articles.

Continued use of products containing chlorhexidine for long periods can cause stains on teeth, the tongue, and gingiva, also on silicate and resin restorations; prolonged use can also reduce bitter and salty taste sensations—this latter symptom can be reversed by ceasing use of chlorhexidine. The brownish discoloration of teeth and tongue are due to the fact that the disintegration of bacterial membranes leads to the denaturation of bacterial proteins. At the same time, disulfide moieties are reduced to thiol moieties that form dark complexes with iron (III) ions found in saliva. Other discolorations might be caused by monosaccharides such as glucose and fructose that are dissolved in saliva and that react with the amine functions of bacterial proteins (Maillard reaction).

A version which stains the teeth less has been developed. The assumption that the extent of discolorations is directly proportional to the efficacy of products containing chlorhexidine is doubtful, due to several reasons. As long as chlorhexidine is incorporated into the bacterial membrane and its substantivity is not impaired, the efficacy of these products should not be affected. Indeed, efforts to prevent the formation of brownish deposits by the addition of reducing agents such as ascorbic acid that react with iron (III) ions, and of nucleophiles such as sulfite ions that react with glucose and fructose, have been successful. Clinical studies with patients suffering from periodontitis show that the post-operative treatment with an ethanol-free mouthrinse containing chlorhexidine (0.2%) for seven days is not negatively affected by addition of ascorbic acid and sulfite (anti-discoloration system ADS®) while the extent of the discolorations observed is lowered significantly. However, a clinical study with healthy volunteers that examined not gingival health but several plaque parameters indicates superiority of a conventional formulation. This apparent superiority is attributed to the ethanol contained in the conventional solution. Moreover, it is assumed that ascorbic acid and sulfite in the ethanol-free mouth rinse prevent the adsorption of the chlorhexidine by teeth and gingiva resulting in a lower substantivity. However, there is no plausible mechanism for such an impairment. The neutral ascorbic acid or the negatively charged ascorbate or the negatively charged sulfite should not affect the attachment of the two-fold positively charged chlorhexidine to teeth and gingiva. Also, a combination of negatively charged sulfite or ascorbate and positively charged chlorhexidine leading to a precipitate of chlorhexidine-sulfite or chlorhexidine-ascorbate does not take place as this would lead to a complete inactivation of the mouth rinse that was never observed. Therefore, it can be concluded that the substantivity of chlorhexidine remains unaffected by the addition of sulfite and ascorbic acid. The apparent inconsistency of the gingival health study with the plaque-regrowth study might be due to differences in the choice of study parameters. While plaque seems to be a required prerequisite for gingival inflammation (gingivitis), a plaque-regrowth study with healthy volunteers, strictly speaking, does not allow conclusions regarding the efficacy of a mouth rinse on the gingival health of patients suffering from periodontitis. However, the gingival health study should be decisive for the dentist in the field.

According to Colgate, chlorhexidine gluconate has not been proven to reduce subgingival calculus and in some studies actually increased deposits. When combined with xylitol, a synergistic effect has been observed to enhance efficacy. Chlorhexidine is neutralized by common toothpaste additives such as sodium lauryl sulfate (SLS) and sodium monofluorophosphate (MFP). Although data are limited, to maximize effectiveness it may be best to keep a 30-minute to 2-hour interval between brushing and using the mouthwash.

In order to increase efficacy and stability, despite possible health risks such as cancer, mouth rinses with chlorhexidine often contain 6-7% ethanol as a preservative; however, ethanol-free chlorhexidine mouthwashes are available in Europe.

Chlorhexidine is used as a topical antiseptic skin scrub in hospital and household settings. It is used for general skin cleansing, as a surgical scrub, and as a pre-operative skin preparation. It is often used as a rubbing agent prior to the use of hypodermic or intravenous needles in place of iodine. Chlorhexidine is contraindicated for use near the meninges, in body cavities, and near the eyes and ears. At the 2% concentration, it can cause serious and permanent injury with prolonged contact with the eye or if instilled carefully and going through the nose through a perforated eardrum. Nevertheless, a topical solution of 0.02% chlorhexidine is recommended by the U.S. Centers for Disease Control and Prevention (CDC) as treatment for keratitis caused by Acanthamoeba. As a scrub, chlorhexidine is not recommended on persons under two months of age. Anionic ingredients in many leave-on topicals and cosmetics, including those in acne products, cleansers, and moisturizers, will inactivate it.

For use in animals, chlorhexidine is used as a topical disinfectant of wounds. Some common brand names are ChlorhexiDerm, ResiChlor, Savinox plus (Bioshields), Germi-STAT Antimicrobial Skin Cleanser, Nolvasan Skin and Wound Cleaner, and Nolvasan Ointment. It is also more beneficial to wound healing than using saline solutions alone. Problems including deafness have been associated with the use of chlorhexidine products in cats. It is commonly used to manage skin infections in dogs. In addition to this it is an active ingredient in teat disinfectant products used within the dairy farming industry.

B. Other Antimicrobials

The use of other anti-microbials is contemplated, including tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, moxifloxacin, metronidazole benzoate, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, teromyocin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, dichlorobenzylalcohol, povidone, sugars, mucopolysaccharides, chlorobutanol, quarternary ammonium compounds such as benzalkonium chloride, organic mercurials, parahydroxy benzoates, aromatic alcohols, halogenated phenols, sorbic acid, benzoic acid, dioxin, EDTA, BHT, BHA, TBHQ, gallate esters, NDGA, tocopherols, gum guaiac, lecithin, boric acid, citric acid, p-Hydroxy benzoic acid esters, propionates, Sulfur dioxide and sulfites, nitrates and nitrites of Potassium and Sodium, diethyl pyrocarbonate, Sodium diacetate, diphenyl, hexamethylene tetramine o-phenyl phenol, and Sodium o-phenylphenoxide, etc. When employed, antimicrobial agent will typically represent from about 1 to about 12 weight percent of the bone cement composition, calculated prior to forming the shaped material.

### IV. BONE CEMENT APPLICATIONS

The presently disclosed TBSCs (those including CaP in their formulation) find use in a variety of clinical (e.g., orthopedic) contexts. As such, they must meet the constraints of medical pharmaceuticals, as discussed below.

A. Bone Cement Compositions

Bone cements refer to entities and compositions that do not produce toxic, allergic, or otherwise adverse reactions when administered to an animal, or a human, as appropriate. In addition to the active agents, supplementary ingredients can also be incorporated into the compositions. For human administration, all preparations should meet sterility, pyrogenicity, general safety and purity standards as required by FDA Office of Biologic Standards.

The compositions are generally formulated for parenteral administration, e.g., formulated for injection. As such, it is necessary that acceptable injectability exists. Upon formulation, solutions will be administered in a manner compatible with the dosage formulation and in such amount as is therapeutically effective.

The actual dosage amount of a composition provided to an animal or human patient can be determined by physical and physiological factors of the patient, as well as the particular application for which the composition is being used. The practitioner responsible for treatment will, in any event, determine the exact type, amount and disposition of the composition per individual subject.

The compositions should be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

B. Uses

While in now way limiting of the various applications for two-solution bone cements of the instant application, the following uses are provided as exemplary of the multiple medical applications for which they can be used.

Arthroplasty. Arthroplasty (literally “surgical repair of joint”) is an orthopedic surgery where the articular surface of a musculoskeletal joint is replaced, remodeled, or realigned by osteotomy or some other procedure. It is an elective procedure that is done to relieve pain and restore function to the joint after damage by arthritis or some other type of trauma.

Previously, a popular form of arthroplasty was interpositional arthroplasty with interposition of some other tissue like skin, muscle or tendon to keep inflammatory surfaces apart or excisional arthroplasty in which the joint surface and bone was removed leaving scar tissue to fill in the gap. Other forms of arthroplasty include resection(al) arthroplasty, resurfacing arthroplasty, mold arthroplasty, cup arthroplasty, silicone replacement arthroplasty, etc. Osteotomy to restore or modify joint congruity is also an arthroplasty.

For the last 45 years the most successful and common form of arthroplasty is the surgical replacement of arthritic or destructive or necrotic joint or joint surface with prosthesis. For example a hip joint that is affected by osteoarthritis may be replaced entirely (total hip arthroplasty) with a prosthetic hip. This would involve replacing both the acetabulum (hip socket) and the head and neck of the femur. The purpose of this procedure is to relieve pain, to restore range of motion and to improve walking ability, thus leading to the improvement of muscle strength. By cementing in implants, not only is the implant fixed to the surrounding bone but a stress gradient is created, which prevents bone loss due to resorption.

Indications include osteoarthritis (OA), rheumatoid arthritis (RA), avascular necrosis (AVN) or osteonecrosis (ON), congenital dislocation of the hip joint (CDH), hip dysplasia (human), acetabular dysplasia (shallow hip socket), frozen shoulder, loose shoulder, traumatized and malaligned joint, joint stiffness.

Vertebroplasty is typically performed by a spine surgeon or interventional radiologist. It is a minimally invasive procedure and patients usually go home the same or next day as the procedure. Patients are given local anesthesia and light sedation for the procedure, though it can be performed using only local anesthetic for patients with medical problems who cannot tolerate sedatives well. During the procedure, bone cement is injected with a biopsy needle into the collapsed or fractured vertebra. The needle is placed with fluoroscopic x-ray guidance. The cement quickly hardens and forms a support structure within the vertebra that provides stabilization and strength. The needle makes a small puncture in the patient's skin that is easily covered with a small bandage after the procedure.

Kyphoplasty is a variation of a vertebroplasty which attempts to restore the height and angle of kyphosis of a fractured vertebra (of certain types), followed by its stabilization using injected bone cement. The procedure typically includes the use of a small balloon that is inflated in the vertebral body to create a void within the cancellous bone prior to cement delivery. Once the void is created, the procedure continues in a similar manner as a vertebroplasty, but the bone cement is typically delivered directly into the newly created void. In its review of vertebroplasty and vertebral augmentation procedures, Medicare contractor NAS determined that there is no difference between vertebroplasty and kyphoplasty, stating, “No clear evidence demonstrates that one procedure is different from another in terms of short- or long-term efficacy, complications, mortality or any other parameter useful for differentiating coverage.”

Fracture augmentation. Fracture augmentation is the process by which bone cement is introduced via injection to stabilize bones that have undergone a fracture. Two of the most common forms of fracture augmentation—vertebroplasty and kyphoplasty—are similar medical spinal procedures in which bone cement is injected through a small hole in the skin (percutaneously) into a fractured vertebra with the goal of relieving back pain caused by vertebral compression fractures.

Bone void filling. Bone void fillers are injectable or moldable compositions that can be flowed or molded into a bone defect, such as a crack, fissure, gap or the like, such as a gap between a synthetic implant (such as a metal prosthesis) and a bone and where bone regeneration is desirable. Examples include the repair of a simple fracture, compound fracture or non-union, external or internal fixation, joint reconstruction, a cosmetic procedure, and the repair or replacement of the bones such as ethmoid, sphenoid, frontal, nasal, occipital, parietal, temporal, mandible, maxilla, orbital, zygomatic, cervical vertebra, thoracic vertebra, lumbar vertebra, sacrum, coccyx, rib, sternum, clavicle, scapula, humerus, radius, ulna, carpal bones, metacarpal bones, phalanges, ilium, ischium, pubis, femur, tibia, fibula, patella, calcaneus, talus, tarsal bones and/or metatarsal bones.

### V. EXAMPLES

The following examples are included to demonstrate embodiments. It should be appreciated by those of skill in the art that the techniques disclosed in the examples which follow represent techniques discovered by the inventor to function well in the practice, and thus can be considered to constitute modes for its practice. However, those of skill in the art should, in light of the present disclosure, appreciate that many changes can be made in the specific embodiments which are disclosed and still obtain a like or similar result without departing from the spirit and scope.

### Example 1

### Materials and Methods

Materials. All chemicals were used as received from manufacturers without any further modification. Poly-methyl methacrylate (PMMA) (Monomer-Polymer & Dajac Labs, Trevose, Pa.) (80,000 g/mol) was added to the cement mixture to provide additional viscosity to the mixture. Methyl methacrylate (Fisher Scientific, Waltham, Mass.) was used as the monomer, while N,N-dimethyl p-toluidine (DMPT)(Sigma Aldrich, St. Louis, Mo.) and Benzoyl Peroxide (BPO)(Sigma Aldrich) were used as the activator/initiator of the free radical polymerization reaction, which is the mechanism of polymerization in this particular cement system. Brushite (CaHPO4.2H2O) (Fisher Scientific), and Hydroxyapatite (Ca₅(PO₄)₃(OH)) (HA) (Sigma Aldrich) were used as the calcium phosphate filler additives, while reagent grade chlorhexidine (CHX) (Sigma Aldrich) and chlorhexidine diacetate salt hydrate (CHX^{I}) (Sigma Aldrich) were utilized as the antimicrobial agents within the cement. Chlorhexidine diacetate (Crescent Chemical Co., Inc., Islandia, N.Y.) was used to validate the results of the CHX^{I} experiments.

Cement and Sample Preparation. Preparation of the traditional TSBC cement (referred to as standard TSBC) followed protocols described in previous studies (Hasenwinkel et al., 1999; Hasenwinkel et al., 2002; Rodrigues et al., 2009). Standard composition cartridges (TSBC), brushite-containing TSBC (referred to as Brushite-TSBC), and hydroxyapatite-containing TSBC (referred to as HA-TSBC) were prepared at concentrations of 25% and 50% (wt/wt PMMA) to allow for a range of CaP fillers within the matrix resulting in a range of powder-to-liquid ratios (0.91-1.43). The additives were well dispersed in the polymer phase of the cement prior to mixing with monomer. In each of the compositions investigated 0%, 2%, 4%, and 8% wt/volume reagent grade chlorhexidine (CHX) or 0%, 2%, 4%, and 8% wt/volume chlorhexidine diacetate (CHX^{I}) were incorporated to provide a range of antimicrobial activity. While molding CHX cement pellets (with reagent grade chlorhexidine) for antimicrobial effectiveness testing, it was observed that as the concentration of CHX in the pellets was increased, the pellets were not reaching full polymerization. There was a notable color change within the cement pellets from off-white to yellow. Because of this, the concentration of DMPT and BPO was increased by 80% in each composition which contained reagent grade chlorhexidine in an attempt to examine whether the properties could be recovered.

The antimicrobial was incorporated into the monomer (liquid) portion of the cement prior to mixing with the rest of the cement mixture. This provided a more thorough dispersion of the antimicrobial into the cement matrix. Cements containing no CHX or CHX^{I} (0%) were made to test the effect of the CaP fillers on the compressive strength of the cement pellets. Cements were prepared, with a concentration of 1.25% (w/v) of benzoyl peroxide (BPO) and 0.7% (v/v) of N,N-dimethyl p-toluidine (DMPT), and the 80% increase represented an increase in both initiator and accelerator concentrations. The cartridges were capped and shaken to mix thoroughly before placing them onto a shaker for 18 hours at 150 RPMs. The compositions were then stored at 4° C. for 72 hours prior to testing to allow for the components of the cement mixture to reach full swelling.

Antimicrobial Effectiveness. The antimicrobial proliferation assay performed was adapted from an assay designed for antimicrobial biomaterial effectiveness screening (Bechert et al., 2000; Alt et al., 2004). Bone cement samples were molded with dimensions of 7×4×3 mm using a PTFE mold. Six samples were molded for each composition tested. Dimensions were selected so that the cement pellets would fit into the wells of a standard sterile 96 well micro-plate. Enterococcus faecalis V583 was selected due to its widespread prevalence in nosocomial infections in hospital settings, as well as its known prolific antimicrobial resistance. Cement pellets were incubated for 1 hour in a new, sterile 96-well microplate with 200 μl of broth culture containing 2×10⁸ CFU of V583, to allow microbial attachment to the cement surface. Another new, sterile 96-well microplate was prepared with 200 μl of 1× PBS solution containing 0.25% glucose, 0.2% (NH4)2SO4, and 1% Brain Heart Infusion (BHI) broth (referred to as 1× PBS+) in each well to be tested. After pellets were incubated in broth culture for 1 hour, they were lightly washed in a 1× PBS solution to remove microbial cells which were not attached to the cement surface. The pellets where then transferred to the next plate containing the 1× PBS+ solution and were incubated for another 18 hours. This step allowed the microbial cells which were attached to the cement surface to release daughter cells into solution without allowing proliferation, as well as replicate a physiologic nutrient poor environment. After the 18 hour incubation period, the cement pellets were removed and 50 μl of BHI broth was added to each well. Cell proliferation was monitored using a kinetic testing procedure on a Monochromater-based Multi-Mode Microplate Reader (Synergy Mx, Winooski, Vt.). The plate was read every 30 minutes for 24 hours after a slow shake step for 5 seconds. After the 24 hour reading step, 1 well of each composition was plated with 100 μl of solution from the well to test for cell viability. Post-cement exposure CFU counting was not performed because the interest of this experiment was to test the cements antimicrobial effectiveness in relation to growth versus no growth.

A single colony of V583 was used to remake a broth culture the day before the proliferation assay was placed into the plate reader. This broth culture was used as a positive control, while an untreated BHI broth culture was selected as a negative control. The antimicrobial experiment was repeated 2 weeks later to validate the results using chlorhexidine diacetate from a different manufacturer. The time necessary to reach an optical density (OD) of 0.2 was standardized as the critical point and defined as the onset OD time for the remainder of the antimicrobial proliferation (Bechert et al., 2000; Alt et al., 2004).

Compressive Strength. A MTS Bionix mechanical testing system (Model 370, MTS Systems Corporation, Eden Prairie, Minn.) was used to perform the mechanical testing experiments to investigate the compressive strengths of the various two-solution bone cements. The effect of CaP fillers was explored by adding HA to one group of compositions and Brushite to another (both in concentrations of 25% and 50% wt/wt PMMA). The effect of chlorhexidine (CHX) and chlorhexidine diacetate (CHX^{I}) incorporation with the cement was also explored by gradually increasing the CHX and CHX^{I} concentration within the cement keeping everything else constant. PTFE molds were used to mold repeatable, symmetrical cylinders for testing. The pellets had dimensions of 6 mm of diameter by 12 mm length. The time lapse between sample molding and mechanical testing was 7 days while the samples were stored at room temperature.

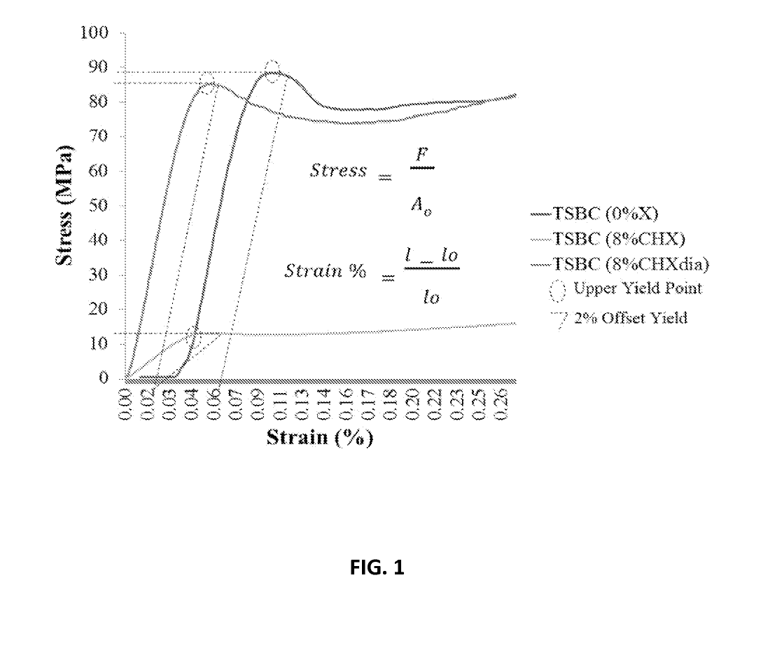

Samples were loaded onto the center ring of compression platens and then subjected to a pre-load of 0.01 kN before beginning the axial ramp at 20 0 mm/min (ASTM Standard F451, 2008). Axial force and axial displacement were recorded and converted to stress and strain using the dimensions of the individual samples. Each composition was tested with 5 samples and the average of these samples was recorded with standard deviations. The compressive strength of the material was defined as the stress at 2% offset from the stress versus strain curve, or the stress at the upper yield point (whichever occurred first) as illustrated in FIG. 1 (ASTM Standard F451, 2008). Samples of cements prepared with an increase in the concentration of BPO and DMPT were tested following the same procedure. The samples were tested 7 days after molding, so the values presented are comparable to the initial data.

### Example 2

### Results

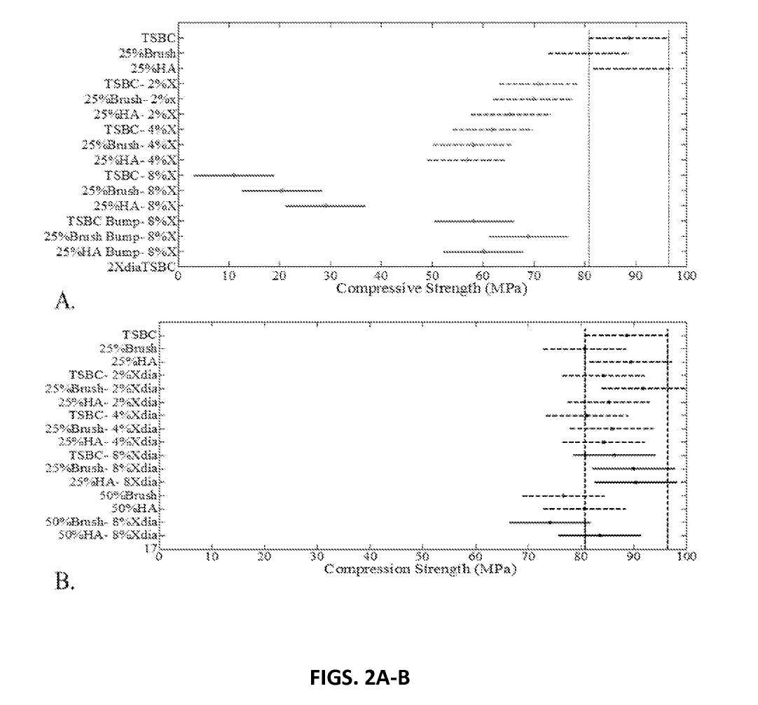

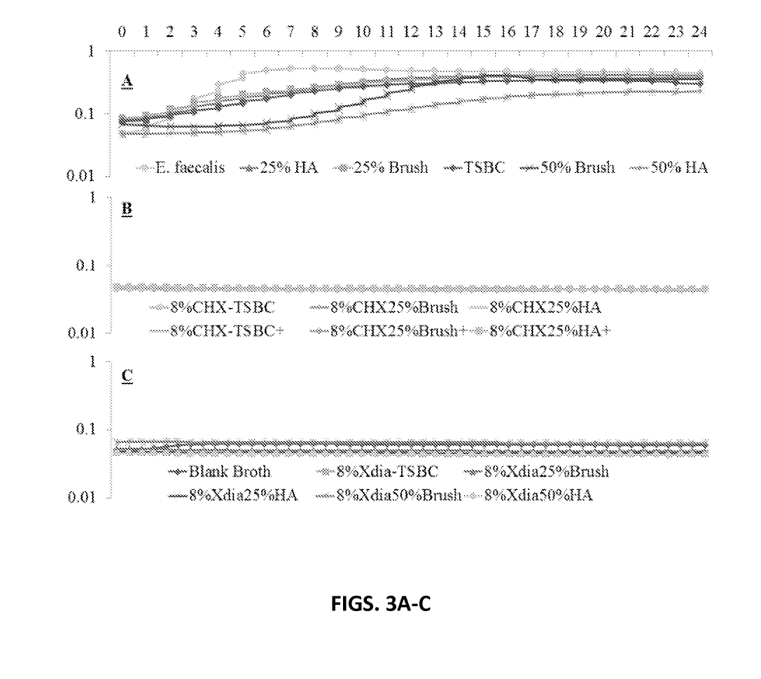

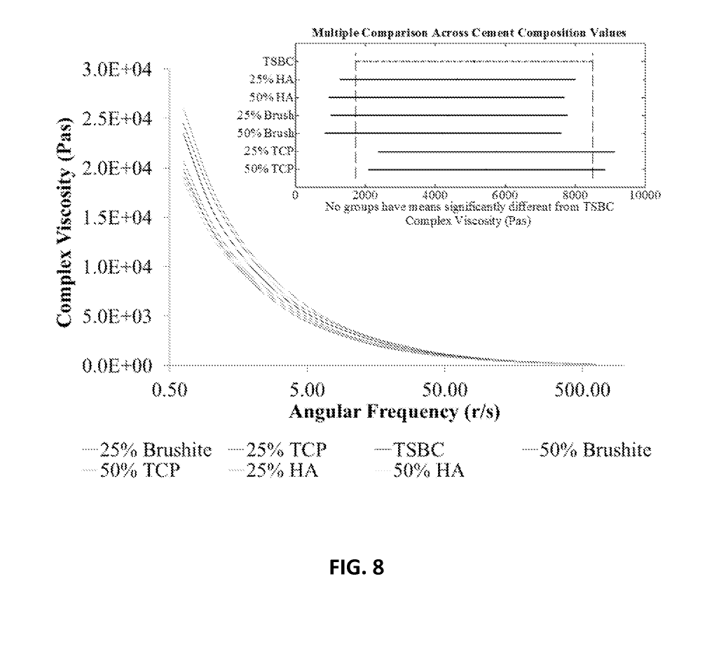

Antimicrobial Effectiveness. The results from the antimicrobial proliferation assay are represented in FIGS. 3A-C. As expected, the TSBC, Brushite-TSBC, and HA-TSBC alone showed no antimicrobial effectiveness without the addition of an antimicrobial, as represented by the standard bacterial proliferation curves (FIG. 3A). As presented in the materials and methods section, the concentration of the CHX and CHX^{I} was increased from 2% to 8% (wt/vol) in each composition to provide a variety of antimicrobial concentrations to investigate. Although, the 2% and 4% (wt/vol) antimicrobial samples displayed a time lag of 4-5 hours prior to onset OD time, they each eventually reached the proliferation log phase, although they plateaued at an average optical density of 0.1 below the positive control. However, each of the 8% (wt/vol) CHX (FIG. 2B) and 8% (wt/vol) CHX^{I} (FIG. 3C) cement compositions demonstrated complete resistance to microbial proliferation in both the original and the validated experiments, as depicted by the flat-lined growth curve plot (FIGS. 3B and 3C). 100 pi aliquots of the 8% (wt/vol) CHX and 8% (wt/vol) CHX^{I} microplate wells were plated on BHI agar plates without dilution to test for cell viability after cement exposure. Each of the plates resulted in bacterial growth.

Compressive Strength: Effect of CaP. The first relationship investigated was the effect of incorporating CaP additives into the cement matrix. Here, Brushite and HA were incorporated into the cement compositions at 25% and 50% (wt/wt PMMA). The compressive strength data illustrated in FIGS. 2A-B and Table 1 demonstrates that the addition of CaP additives at these concentrations with no CHX or CHX^{I} did not significantly change the compressive strength of the cement compositions as compared to the control TSBC cement (p>0.05).

TABLE 1

| Composition | Trial #1 | Trial #2 | Trial #3 | Trial #4 | Trial #5 | Average | St. Dev. | E | St. Dev. |
|---|---|---|---|---|---|---|---|---|---|
| TSBC | 82.45 | 87.14 | 85.51 | 99.86 | 88.41 | 88.60 | 6.64 | 4.97 | 0.40 |
| 8% XdiaTSBC | 83.06 | 90.49 | 85.20 | 81.70 | 91.23 | 86.33 | 4.32 | 5.30 | 0.48 |
| 25% Brushite | 86.54 | 75.11 | 79.37 | 88.32 | 74.29 | 80.73 | 6.45 | 4.80 | 0.60 |
| 8% Xdia25% | 87.79 | 83.99 | 96.25 | 91.20 | 90.35 | 89.92 | 4.51 | 4.96 | 0.52 |
| Brushite | | | | | | | | | |
| 25% HA | 78.55 | 77.40 | 103.71 | 88.90 | 98.75 | 89.46 | 11.77 | 5.05 | 0.71 |
| 8% Xdia25% HA | 84.23 | 93.75 | 92.52 | 91.67 | 89.75 | 90.39 | 3.73 | 6.07 | 0.50 |
| 50% Brushite | 78.96 | 80.07 | 73.80 | 78.44 | 72.39 | 76.73 | 3.41 | 3.15 | 0.57 |
| 8% Xdia50% | 75.53 | 69.81 | 71.28 | 70.13 | 83.82 | 74.11 | 5.89 | 3.04 | 0.51 |
| Brushite | | | | | | | | | |
| 50% HA | 84.11 | 82.56 | 69.99 | 82.34 | 84.39 | 80.68 | 6.04 | 4.91 | 0.84 |
| 8% Xdia50% HA | 93.31 | 86.35 | 85.57 | 70.20 | 82.44 | 83.57 | 8.47 | 5.48 | 0.61 |

Compressive Strength: Effect of CHX. As hypothesized, the addition of CHX caused the compressive strength of the cement compositions to decrease. As the concentration of CHX was increased from 2%-4%-8%, the compressive strength decreased accordingly (FIGS. 2A-B). In each composition tested with CHX incorporated, the compressive strength significantly decreased (results were no longer significantly similar to the control samples) when compared to the control TSBC cement (p<0.05). This relationship was inversely proportional—as the concentration on CHX in the cement was increased, the compressive strength of the cement decreased. To supplement this phenomenon, the concentration of BPO and DMPT was increased by 80%. The 80% increase of BPO and DMPT within the reagent grade chlorhexidine samples was successful in aiding the restoration of the compressive strength of the samples by an average of 49% across the samples tested (FIGS. 2A-B). However, it is clear from the multiple comparison that these values are still significantly lower than the control composition (p<0.05) (FIGS. 2A-B).

Another note observed in testing these samples was the large standard deviations within the 8% CHX samples, as compared to the other samples tested with and without CHX. The 8% CHX cements had standard deviations 20% higher than any other composition tested. This is likely due to the free radical quenching by the CHX molecule being non-uniform.

Compressive Strength: Effect of CHX^{I}. CHX^{I} was explored to investigate whether it maintained this same trend of decreasing compressive strength as concentration was increased in the cement compositions the way reagent grade chlorhexidine did. The results illustrated that the CHX^{I} cements demonstrated no free radical quenching during the polymerization reaction; the cements retained their compressive strength in each composition examined (FIGS. 2A-B). There was no significant difference (p>0.05) between the compressive strengths of the CHX^{I} cement compositions as compared to the control samples regardless of the concentration of antimicrobial in the composition.

Elastic Modulus. All cement compositions investigated resulted in elastic moduli in the range of 3-6 GPa (Table 1), with the 50% Brushite-TSBC compositions resulting in the lowest modulus (in average 3 GPa) with and without antimicrobial addition. The elastic moduli of these Brushite-TSBC were significantly lower than the other cement compositions investigated (p>0.05).

### Example 3

### Discussion

This study was designed to investigate and isolate an optimal composition of two-solution acrylic bone cement for use in orthopedic applications containing both bioactive and antimicrobial properties. The goal was to develop a composition which demonstrated antimicrobial effectiveness and bioactivity while retaining its physical and mechanical properties. The bioactive agents were incorporated at 25% and 50% (wt/wt PMMA) to improve integration of the cement with bone. It was clearly demonstrated in the results (FIGS. 2A-C and 3A-C; Table 1) that the addition of CaP additives at this concentration was not detrimental to the antimicrobial or compressive strength characteristics. A previous investigation on the TSBC material incorporated with CaP fillers illustrated that the material exhibits no filter-pressing or increased extrusion stress as many other CaP filled cements demonstrate (Baroud et al., 2005). Because TSBC material spends a dramatically increased amount of time in the swelling phase, as compared to powder-liquid formulations, it is thus better suited to incorporate additives fully into its polymer matrix.

Chlorhexidine was investigated as a potential antimicrobial for use in the proposed cement formulation. Chlorhexidine is attractive for use in the two-solution cement because of its wide spectrum, lack of microbial resistance, and inexpensive cost compared to other commercially available antibiotics. One major concern was the phenomenon of decreasing compressive strength of the cements while increasing the CHX concentration (Holt et al., 2007; Botelho, 2004; Shahi et al., 2007). It is also reported throughout the dental cements literature and was observed in each of our reagent grade CHX samples investigated in this study.

The initiation chemistry concentrations were increased on the reagent grade chlorhexidine samples because it was hypothesized that the cement was not reaching full polymerization due to the free radical quenching provided by the CHX molecular structure. Vinyl chloride functional groups are known to be attacked by free radicals formed by activation of BPO and propagating polymer chain, as well as the reactive imine groups within the CHX molecule. This interaction was acting as a plasticizer during polymerization of the cement matrix, quenching the free radicals and leading to a premature termination of the free radical propagation. Even, an 80% increase of initiation chemistry was not substantial enough to fully recover the original compressive strength of the control cement (FIGS. 2A-B). There was still a color change within the cement compositions containing CHX upon hardening, indicating that some level of radical quenching was still occurring, just not fast enough to completely stop the polymerization reaction with the increased BPO and DMPT concentration. It was hypothesized that increasing the concentration of BPO and DMPT further could completely recover the compressive strength of these cements. However, additional increases in the initiation chemistry concentrations could lead to other detrimental effects in the cement as reported by Hasenwinkel et al. (Hasenwinkel et al., 2002). Therefore, the 80% increase or any additional increase was deemed not adequate due to color changes and other possible effects.

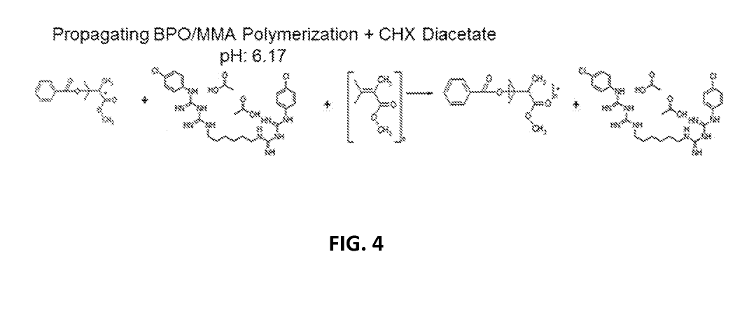

It seems there is a common trend within the literature when incorporating chlorhexidine (either reagent grade or digluconate) into cements; as the concentration of chlorhexidine within the cement compositions is increased, the lower the compressive strength of the cement becomes (Holt, et al., 2007; Botelho, 2004; Shahi, et al., 2007). Although the reagent grade chlorhexidine resulted in decreased compressive strength of the cement compositions, interestingly the use of chlorhexidine diacetate demonstrated identical antimicrobial effectiveness while retaining, and in some cases slightly increasing, the compressive strength quality of the cement (FIGS. 2A-B). The diacetate grade was attempted in order to investigate whether the acetate ion in the composition could shield the quenching reaction, which was taking place in the reagent grade mixture. The proposed mechanism of this interaction is thought to be due to the acetate ion causing an ionic interaction with the CHX molecule and stabilizing it to the attack by the free radical (FIG. 4). This “shielding effect” allows the CHX molecule to remain intact and uncompromised during the polymerization reaction, retaining its antimicrobial qualities. In either case, the retention of compressive strength among the CHX^{I} cements, specifically the 8% CHX^{I} cement compositions, is incredibly substantial due to its resistance to antimicrobial proliferation. Because these cements were shown to be able to retain their mechanical properties, while demonstrating resistance to microbial proliferation, their use in orthopedic applications may be considered. Cements prepared with CHX diacetate maintained compressive strength values above 70 MPa (the minimum compressive strength necessary for implant augmentation applications per ASTM standards (ASTM Standard F451, 2008)). Elution experiments will be performed to determine the amount of time the cement pellets remain antimicrobial.

After being exposed to the CHX cement pellets containing 8% CHX or CHX^{I}, there was no bacterial proliferation within the wells of the microplate (FIGS. 3B-C). However, viability plating after the experiment yielded a lawn of bacteria grown from each well tested indicating the bacteriostatic nature of the cements rather than bacteriocidal. This demonstrated that the cells remain viable in the presence of CHX or CHX^{I} cement environments; however, they are not capable of proliferating when this environment contained CHX or CHX^{I} at 8% (wt/vol). This is a beneficial result, because it suggests that the material is capable of preventing the proliferation of bacteria, while allowing cells in the environment to remain viable. Further toxicity studies will be conducted to ensure the viability of osteoblast and fibrinogen cells in contact with the cement surface.

The elastic modulus of the cement material is a critical characteristic, because moduli which differ significantly from human bone can result in stress shielding. The closer the material modulus is to that of natural bone, the better the material will mimic the bone properties. These materials are better suited to distribute stresses accordingly. Human trabecular and cortical bone are reported to have elastic moduli of approximately 3.8 and 5.4 GPa, respectively (Choi et al., 1990). The 50% Brushite-TSBC compositions were the only materials tested with significantly lower moduli (3.04-3.15 GPa) (p>0.05). All the other compositions were observed to have moduli in the range of other commonly employed commercial cements.

Along with their faster resorption times, and their equivalent antimicrobial effectiveness, the higher concentration Brushite cement compositions developed seem to be the best fit for an antimicrobial and osteoconductive bone cement/substitute. As these materials interact with bone, they are better adjusted to act as natural bone. Further, as they are resorbed, their constituents are available for osteoblasts to utilize in bone reformation and will result in bone replacing the cement matrix over time. Because the cement base is still acrylic in nature, further studies are currently being conducted to replace the acrylic polymer concentration with a higher CaP (Brushite) concentration to allow for increased levels of resorbability and better rheological characteristics (increased zero-shear viscosity and improved injectability). It is further hypothesized that this phenomenon of mechanical strength retention using chlorhexidine diacetate could enable dental cements, like the MTA root-end fillers, to adopt a similar method of antimicrobials within their compositions to allow for load-bearing applications in dental cements.

Although this study demonstrated that CaP fillers and antimicrobials could be incorporated into two-solution bone cements without impairing the material characteristics, further investigation is still needed. This study dealt with chlorhexidine alone. A more comprehensive study will need to be performed to demonstrate the effectiveness of varying amounts and types of antimicrobials in TSBCs. Also, burst effect is always a concern in drug delivery materials. Because the material will be utilized inside of the body, toxicity testing will be necessary to determine the biocompatibility of the material. Osteoblasts, and fibrinogen cell viability testing will be conducted. Finally, this current study used fixed polymer to monomer ratios. The CaP fillers did not replace the polymer concentration in the cement composition. A study to determine the effect of CaP replacement of the acrylic polymer has been initiated.

There is currently little evidence supporting the prophylactic treatment of prosthesis-related infection with systemic antibiotics after surgery (Guvenm & Uzun, 2003). For the prophylactic treatment to be affective, the optimal concentration of antibiotic/antimicrobial must be localized to the primary (potential) infection site. Such localization of antimicrobials could be optimized by the used of antimicrobial loaded bone cements. Until now the use of CHX in cements has been limited due to the observed detrimental effects on the materials properties. This study showed the CHX diacetate might be a potential antimicrobial for use in cements. Because the addition of the acetate within the CHX (chlorhexidine diacetate) allows for the complete polymerization of the cements, the cement compositions are capable of retaining their mechanical properties while remaining antimicrobial. This observation may allow for the use of chlorhexidine-containing cements in load bearing applications for the first time. HA is known to mimic the scaffold of cancellous bone (Jones & Hench, 2003); however, brushite's Ca:P ratio provides it with a much faster and more complete resorption within physiologic environments (Tamimi, et al., 2012; Grover, et al., 2003). Therefore, a HA-TSBC composite could provide superior stabilization of compressed osteoporotic or diseased vertebrae, providing venues for biological interlocking, while a highly resorbable Brushite-TSBC could provide a scaffold for bone growth and integration with the remaining cement matrix, being adequate for augmentation of trauma-related fractures in a younger patient population.

### Example 4

### Materials and Methods

Materials. All chemicals were used as received from manufacturers without any further modification. PMMA (Monomer-Polymer & Dajac Lab, Trevose, Pa.) (80,000 g/mol) was used as an aid to increase the viscosity of the cement mixture. Methyl methacrylate (Fisher Scientific, Waltham, Mass.) was used as the monomer for the mixture. N,N-dimethyl p-toluidine (DMPT) (Sigma Aldrich, St. Louis, Mo.) and Benzoyl Peroxide (BPO)(Sigma Aldrich) were used as the activator/initiator of the free radical polymerization reaction. HA (Sigma Aldrich), Brushite (Fisher), and TCP (Sigma Aldrich) were used as the calcium phosphate filler additives in the cements.

Cement Preparation. The preparation of the standard TSBC followed protocols described in previous studies (Josefsson et al., 1981; Lautenschlager, et al., 1976). Standard composition cartridges (TSBC), hydroxyapatite-containing TSBC (referred to as HA-TSBC), brushite-containing TSBC (referred to as Brushite-TSBC), and TCP-containing TSBC (referred to as TCP-TSBC) were prepared with a fixed polymer (PMMA) to monomer (MMA) ratio (P:M) of 0.9:1. Non-setting cements were used to investigate the effect of CaP filler addition on the viscosity of the cements in the doughy phase. This eliminated variations in viscosity due to the complex cement curing process¹⁸. Setting cements were also prepared for injectability assessment and gel point determination. Setting cements were prepared, with a concentration of 1.25% (w/v) of benzoyl peroxide (BPO) and 0.7% (v/v) of N,N-dimethyl p-toluidine (DMPT). HA, Brushite and TCP were added at concentrations of 25% and 50% (wt/polymer wt) in relation to the total PMMA weight. 50% (wt/polymer wt) CaP was selected as the limit for CaP additive addition because the CaP additives were not replacing any polymer in the mixture, and thus were not affecting the polymer to monomer ratio. The additives were well dispersed in the polymer phase of the cement prior to mixing with monomer. The cartridges were capped and shaken to mix thoroughly before placing them onto a shaker for 18 hours at 150 RPMs. The compositions were then stored at 4° C. for 72 hours prior to testing to allow for the components of the cement mixture to reach full swelling. The final powder-to-liquid ratio (P:L) of the cement compositions were 0.91 (TSBC), 1.13 (25% CaP), and 1.35 (50% CaP).

Rheology of Non-Setting Cements. Rheology tests were performed using a Discovery HR-3 hybrid rheometer (TA Instruments, New Castle, Del.). All cement compositions were removed from 4° C. and allowed to reach room temperature for 2 hours prior to testing as per ASTM Standard F451-08 (ASTM Standard F451, 2008). Static mixing nozzles (Ellsworth Adhesives, Germantown, Wis.) were used to mix the two components of the material and elute the mixture onto the bottom rheometer plate. Given the size of the filler materials used in the cement mixture, disposable parallel plate geometries were selected for this study. Parallel plates with a diameter of 25 mm were used with all cement compositions evaluated. Material volumes of 0.5 mL and a geometry gap of 1000 μm +/−300 μm were set as testing standards. Materials were also tested at a 1 mL volume and 2000 μm geometry gap to ensure the data was not dependent on dimensional parameters. After examination at 2000 μm and a 1 ml sample volume, it was determined that the measurements were independent of this variable and the 1000 μm geometry gap with 0.5 ml sample volume were adopted for the remaining samples throughout the test. Samples were loaded at a fixed gap of approximately 45,000 μm and the geometry was set to a final gap of 1000 μm +/−300 μm for testing after trimming the sample at 1050 μm.

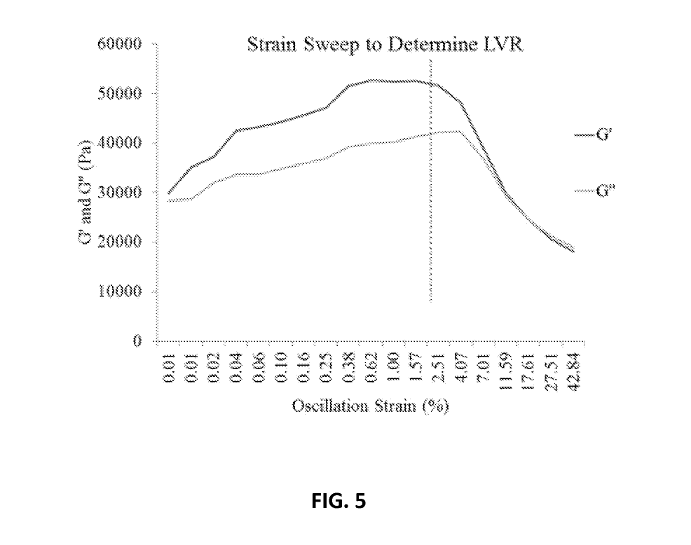

Oscillation frequency was selected over continuous shear due to the viscoelastic behavior of the material and observation of a Weissenberg effect in steady shear stress mode. This is a common finding when rheologically characterizing materials such as bone cements. During steady shear, the material may climb up the geometry, resulting in edge failure (Weissenberg effect). Dynamic rheometry was thus adopted as the means of characterizing the material properties. The testing parameters remained constant for each composition tested. Testing temperature was set to 24° C. with no soak time. Continuous oscillation frequency was applied using a logarithmic sweep function with a frequency range of 100-0.1 Hz. Percent strain was constrained to 0.5% for each test after an amplitude sweep was performed to detect the range of the linear viscoelastic region (LVR). It was observed that the material reached the non-linear viscoelastic region (critical strain) at approximately 5% oscillation strain, which prompted operation at 10% below the critical strain (FIG. 5).

Data was validated using a separate operator, as well as adding a conditioning step prior to the frequency ramp. The conditioning step used a 0.5% strain and 10 rad/sec frequency over 30 seconds. Data from the validation experiment coincided with the original data. Parameters investigated included storage and loss modulus (G′ and G″), complex viscosity (η*), and time. Table 2 summarizes the dynamic rheology equations used to obtain the parameters of interest.

TABLE 2
Dynamic Rheological Parametrs and Equations

| Parameter | Representation | Equations |
|---|---|---|
| γ | Shear Strain (%) | |
| γₒ | Maximum Shear Strain | γ = γₒ sin ωt |
| ω | Angular Frequency | |
| t | Time | |
| σ | Oscillatory Stress | σ = σₒ sin(ωt + Ø) |
| σₒ | Original Osc. Stress | |
| Ø | Phase Angle | |
| η* | Complex (True) Viscosity | η* = σ/(γω) |
| η′ | Real (Elastic) Component of | η* = η′ − iη″ |
| | Complex Viscosity | |
| η″ | Imaginary (Viscous) Component of | |
| | Complex Viscosity | |
| G* | Complex Modulus | G* = G′ + iG″ |
| G′ | Real (Elastic) Component of | |
| | Complex Modulus | |
| G″ | Imaginary (Viscous) Component of | |
| | Complex Modulus | |
| i | −1^{1/2} | |

Rheology of Setting Cement: Gel Point Analysis. The rheology of setting cement compositions was investigated to demonstrate the effect of CaP additives on the setting characteristics (setting time) of the cement. The materials were tested using a similar procedure as previously described for the non-setting cement compositions. Here, the rheometer and loading procedure remained the same, but the material was investigated over time at a constant angular frequency of 10 rad/sec. Mixing nozzles were used to extrude the material from the cartridge while thoroughly mixing the two components. Once ejected from the mixing nozzle, the content of the two cartridges are thoroughly incorporated and the polymerization reaction will commence. Storage and loss moduli of the material were investigated to find the materials gel point. The gel point was defined as the point at which the storage modulus equals the loss modulus (moduli crossover) as shown in Equation 1:

G′G″ (1)

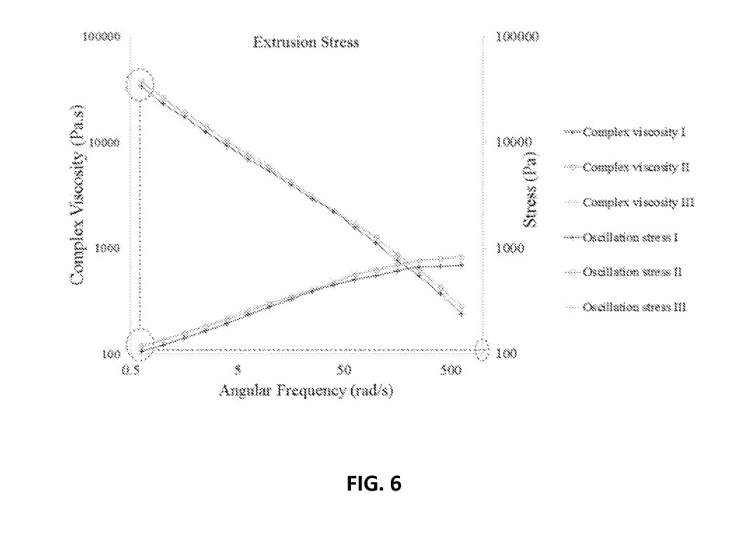

Extrusion Stress (σ^{extrusion}). Yield stress is an important parameter because it gives an estimate of the pressure or stress required for injection or extrusion of a material. However, pseudoplastic materials demonstrate no true yield stress, meaning they are capable of flowing instantaneously following stress application. Therefore, they are defined as behaving in a non-Newtonian manner due to their display of shear-thinning effects. Thus, the extrusion stress (σ^{extrusion}) of each of the formulations developed was investigated. The extrusion stress was defined as the oscillatory stress value recorded at the maximum viscosity (lowest angular frequency which elicited an oscillatory stress, at 0.63 rad/sec) of the material during frequency sweep as illustrated in FIG. 6. Because this oscillatory stress refers to the stress at the maximum viscosity of the cement composition, it is directly related to the maximum stress required to begin extrusion of the cement mixture from its cartridge.

Injectability. The purpose of the injectability test was to determine the efficacy of extruding the cement through the cartridge-nozzle system. To ensure repeatability, a pneumatic gun (Ellsworth, Germantown, Wis.) was used at maximum pressure (5.2 Barr) to push the cement through the system. A fresh cartridge of cement was used for each trial conducted. Cement was pushed out a double barrel cartridge connected to a mixing nozzle. The time from when cement exited the tip of the nozzle to when a 1 mL Eppendorf vial was filled was recorded and converted into an injectability rate of milliliters per minute.

Imaging. The surface morphology of TSBC, HA-TSBC, Brushite-TSBC, and TCP-TSBC was investigated using a Keyence VHX-2000 Digital Microscope (Osaka, Japan). All images were obtained from dry cement samples 24 hours post-polymerization and stored at 25° C. open air environment. For imaging of the cement matrix, each sample was molded using a polytetrafluoroethylene (PTFE) mold, allowed to fully polymerize, and then broken in half. This way, the imaging could take place within the cement matrix rather than at the molded surface. All imaging was performed using identical parameters to ensure proper comparison among the different compositions surveyed.

Statistical Analysis. A one-way Analysis of Variance (ANOVA), as well as a multiple comparison across values tests were performed to show differences among the groups compared at a confidence interval of 95% using MATLAB (R2012a, Natick, Mass.). The multiple comparisons performed provided information about which pairs of means were significantly different from each other, and which were not. The multiple comparisons across values test utilizes a one-way ANOVA test to compare means of data to a control group. In this way, the p-value of the ANOVA could be validated and the means of data could be compared to the control.

### Example 5

### Results

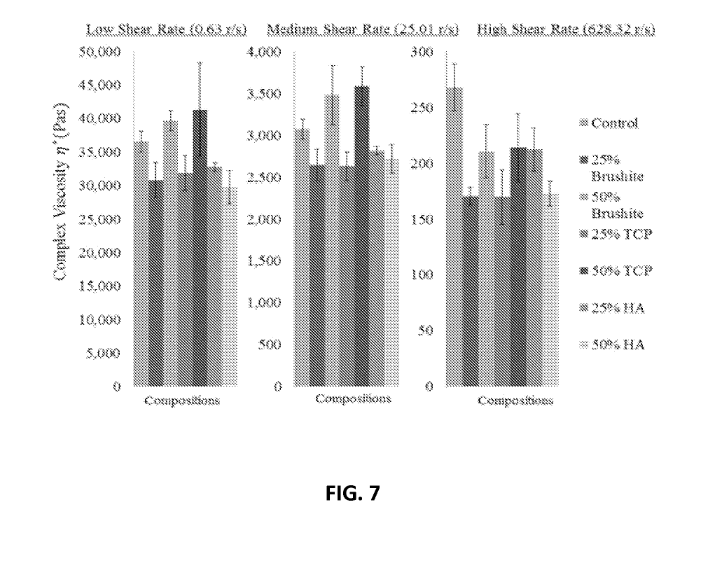

Rheology of TSBC containing CaP fillers. Rheological characterization using the techniques previously described revealed no significant effect (p>0.05) of the addition of the three CaP fillers investigated in the two-solution bone cement formulation. HA-TSBC, TCP-TSBC and Brushite-TSBC were all prepared at the same concentrations of CaP fillers and the rheological behavior was compared to the standard TSBC (prepared with no fillers). The additions of 25% (wt/polymer wt) of the calcium phosphate substitutes investigated resulted in an average decrease of approximately 3600 Pa·s at low shear rates (0.63 rad/s), approximately 240 Pa·s decrease when performing the test at medium shear rates (25.00 rad/s), and 35 Pa·s decrease at high shear (628.0 rad/s), as illustrated in FIG. 7. Further, although 50% (wt/polymer wt) additions of Brushite and TCP resulted in an average increase in viscosity of approximately 1200 Pa·s, 50% (wt/polymer wt) HA demonstrated an average decrease in viscosity of 1100 Pa·s. This decrease and/or maintenance of cement viscosity with high concentration of CaP fillers is advantageous because it correlates to improved injectability characteristics of the compositions as well as the maintenance of the pseudoplastic nature of the material.

The TSBC system is advantageous because of the high pseudoplastic behavior which results in improved injectability. It is evident from FIG. 7 that the addition of the three calcium phosphate fillers did not reduce pseudoplasticity of the cements, as revealed by the negative slopes in comparison to the control formulation (Table 3). The slopes of the regression lines shown in FIG. 8 and Table 3 give the type and degree of non-Newtonian flow, in which a zero slope would imply Newtonian behavior. Thus, the shear thinning effects are uncompromised by the addition of CaP additives in TSBC. FIG. 8 shows that as the material is subjected to shearing, its viscosity is greatly reduced. This means the material is capable of being extruded from mixing nozzles and delivered to the location of interest, while upon removal of pressure, or at lower shear rates, the viscosity will recover exponentially.

TABLE 3
Rheological Characteristics of CaP-TSBC

| | Slope of | | |
|---|---|---|---|
| | Dynamic | | Average |
| | Viscosity | Extrusion Stress | Injectability |
| Composition | Curve | (σ^{extrusion})(Pa) | Rate (ml/min) |
| Control TSBC | −0.77 | 115.47 | 1.13 |
| Hydroxyapatite (25%) | −0.73 | 107.02 | 2.37 |
| Hydroxyapatite (50%) | −0.74 | 97.92 | 1.58 |
| Brushite (25%) | −0.74 | 102.29 | 1.20 |
| Brushite (50%) | −0.74 | 123.77 | 0.58 |
| Tricalcium Phosphate | −0.75 | 103.76 | 2.50 |
| (25%) | | | |
| Tricalcium Phosphate | −0.75 | 133.83 | 2.63 |
| (50%) | | | |

The multiple comparisons across composition values depicted in FIG. 8 illustrate that although there are slight variations from composition to composition, when compared against a control group, there is no significant change in values of each group's means at each data point analyzed at a 95% confidence interval. Because of this, it can be deduced that compositions of 25% to 50% (wt/polymer wt) CaP additives in TSBC showed no significant change in the rheological properties of each composition compared to the control group samples.

Gel Point. The gel point of each cement composition was investigated to observe if the CaP fillers would result in a change in the setting time of the cements. This experiment was not meant to substitute the standard ASTM exothermal test for bone cements, from which maximum polymerization temperature and setting time are obtained using a standard mold (ASTM Standard F451, 2008). The goal was to observe changes in this parameter with filler addition and filler concentration. Each composition investigated resulted in similar gel point values of approximately 700 kPa (G′G″); however the time to reach the gel point was not equal in each trial. Each composition tested reached its gel point between 185-260 seconds; however, after the addition of each additive, the time to reach gel point was less than that of the control group by an average of 44 seconds. This is likely due to the slight variations, in the loading time for each sample (1.5 min+/−0.5 min), as well as sample size (0.5 ml+/−0.15 ml). It was observed that the addition of CaP fillers significantly affected the gel point time of all cement compositions (p<0.05) in comparison to the standard TSBC. Thus, the addition of CaP additives to the TSBC cement compositions did significantly reduce the gel point time of the cements irrespective of the concentration of the additives, between 25-50% (wt/polymer wt).

Extrusion Stress (σ^{extrusion}). The extrusion stress of the TSBC cement control as well as the additive compositions ranged from 98-134 Pa (Table 3). Although powder-liquid formulations undergo a dramatic change in viscosity from liquid-like to viscous-like mixtures, while TSBC begins the mixing process in the doughy state, these reported extrusion stresses are consistent with reported yield stress values of some commercially available powder-liquid formulations (Qingsong & Salovey, 2004). It is important to note that there is no significant change in extrusion stress after up to 50% (wt/polymer wt) CaP additive incorporation within the cement mixture (p<0.05).

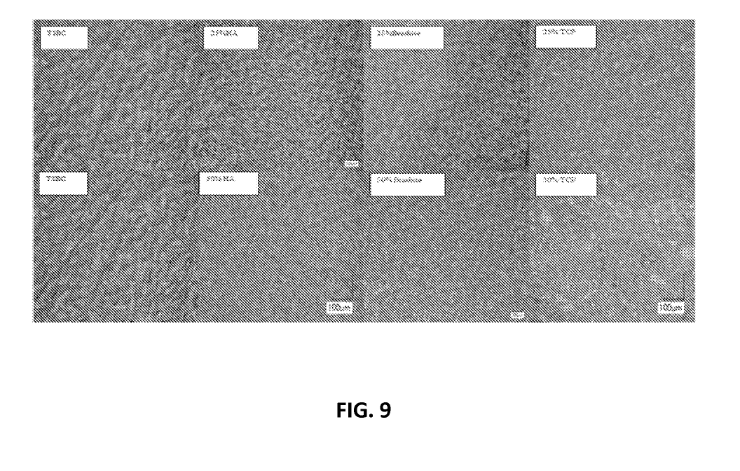

Injectability. The injectability data in Table 3 shows that the addition of bioactive fillers in the TSBC does not cause any hindrance during injection. Excluding the 50% Brushite composition, the other samples demonstrated improved injectability compared to TSBC. However, the shear-thinning effect is not affected by the filler. As the concentration of the filler is increased from 25% to 50% (wt/polymer wt), there is still no detrimental effect on the injectability rate, except in 50% (wt/polymer wt) Brushite. However, FIG. 9 illustrates that the addition of the filler phase in TSBC did not result in clump formation, fully incorporating and dispersing within the cement matrix.

### Example 6

### Discussion

Calcium phosphate compounds were incorporated in a two-solution cement system to overcome the lack of bioactivity observed in a standard formulation. The ultimate goal of this research was to produce a unique bioactive, high-viscosity, and easily injectable PMMA two-solution bone cement for multiple uses in orthopedic surgery. This cement combines the mechanical strength of acrylic PMMA with the benefits of CaP compounds. Because CaP fillers are known to detrimentally degrade bone cement handling and injectability (Yang et al., 2012; Low et al., 2010), it is critical to evaluate the effect of such fillers in the rheological characteristics and injectability when developing new formulations of bioactive cements. HA, TCP and Brushite were selected for addition in TSBC because they exhibit particular stability and resorption rates, which can be beneficial for different applications and patient needs. For example, a HA-TSBC is not expected to display significant mass loss because of the high calcium-to-phosphate ratio (Ca:P) of HA, which would ensure bioactivity with very slow resorption rates; whereas a Brushite-TSBC would be resorbable due to the low Ca:P ratio of Brushite.

It has been demonstrated in this study that addition of HA, TCP and Brushite into TSBC alone does not detrimentally affect the viscosity and good injectability properties of the two-solution cement system. In fact, a slight reduction in viscosity was observed for some of the compositions studied, as illustrated in FIG. 7. The injectability data shown in Table 3 illustrated that the injectability rate of the cements is increased with added CaP fillers up to 50% (wt/polymer wt) (with the exception of the Brushite composition). Analysis of matrix morphological features showed that the cements displayed no clumping or particle agglomeration in any composition up to 50% (wt/polymer weight). This suggests that although the injectability rate may have been slower, filter pressing and clogging is still a non-issue with these compositions (Baroud et al., 2005). These rheological characteristics are believed to be a result of the improved dispersion and full polymer swelling achieved by the two-solution system. There is an agreement within the literature that smaller particle sizes result in increased viscosity [20] in polymer systems because they tend to form particle networks that produce a yield phenomenon (Qingsong & Salovey, 2004). However, the more dispersed the particles, the lower the viscosity of the cement. When adding CaP fillers in bone cements, several events can lead to an increase in viscosity and degradation of the material handling including: (1) increased particle volume in the mixture can lead to difficult wetting of the components of the cement mixture; (2) particle-particle interaction and network formation may lead to clumping of the filler phase; and (3) phase separation or filter-pressing. To overcome some of the drawbacks provided by the addition of CaP fillers in powder-liquid cements, (Bohner & Baroud, 2005) also suggested an increase in the L:P ratio and decrease in the plastic-limit (PL) of the powder besides decreasing particle size (Bohner & Baroud, 2005).

In current powder-liquid calcium phosphate cements, an ionic modifier (such as sodium polyacrylate) may be used as a dispersant of the calcium phosphate particles to allow for improved injectability characteristics. The addition of such an ionic modifier allows for an increase in the electrostatic repulsive forces between the different particles in the formulation (Baroud et al., 2005). However, an increase in the liquid-to-powder ratio is not recommended because it can induce significant changes in the cement thermal properties and residual monomer levels. It is important to note that no dispersant agents or fluctuation in milling time was necessary in the TSBC formulations. Also, none of the TSBC cements exhibited phase separation during extrusion. Additives may resolve the injectability problems associated with powder-liquids cements containing high loads of CaP, however they can trigger changes in other properties such as mechanical integrity, thermal and setting behavior (Bohner & Baroud, 2005).

It was demonstrated that the addition of CaP additives to the TSBC resulted in a decrease in the time to reach gel point within the cement (setting time). However, one of the benefits of the TSBC system is that the setting time of the material can be easily adjusted by variations in the concentration of initiator and accelerator (Hasenwinkel et al., 2002). The extrusion stress values of each TSBC composition tested was within the normal range of yield stress values for acrylic bone cements (Qingsong & Salovey, 2004). This is a critical characteristic because it relates to the stress or pressure required to extrude or pump a material from its delivery system. Because of its prolonged swelling time within the delivery cartridge, the TSBC reaches its doughy state prior to extrusion. Despite this, it is still within the normal range of stress values at its maximum viscosity.

Another important observation was the high shear-thinning nature of TSBC containing CaP fillers. FIG. 7 illustrates that the pseudoplastic nature of TSBC was unaltered by either concentration of CaP fillers added, as seen by the slopes of the complex viscosity data. This is an important property because it will ensure ease of injection of highly viscous cements, such as the TSBC compositions investigated. High viscosity cements are desirable to stabilize cement flow, minimizing the risk of leakage (Baroud et al., 2006). However, the challenge is that highly viscous cements will require forces for delivery that may approach or even exceed the human physical capability for injection (Chen et al., 2011). The pseudoplasticity of HA-TSBC, TCP-TSBC and Brushite-TSBC facilitates flow through surgical needles or cannulas because the viscosity shear thins while pressure is provided, undergoing quick recovery once pressure is removed. TSBC prepared with cross-linked nanoparticles and microparticles were demonstrated in a previous study to have high pseudoplastic behavior, and therefore, suitable properties for applications in the treatment of vertebral compression fracture (Rodrigues et al., 2009). Because the viscosity of these TSBCs is recovered once the cement is delivered to the treatment site, the risk for extravasation is minimized, which could potentially improve the outcomes of fracture treatment via bone cement.

Experimental results illustrated that the time to extrude each cement evaluated was significantly lower than the setting time, allowing the materials to interdigitate well with the bone pores before setting. At a 25% (wt/polymer wt) concentration, all additives increased the injectability rate in comparison to the standard, while at 50% (wt/polymer wt) CaP additives showed no trend in injectability. The injectability rate for 50% (wt/polymer wt) Brushite-TSBC was half that of the control (TSBC), while the injectability rate for 50% (wt/polymer wt) TCP-TSBC was slightly faster than that of 25% (wt/polymer wt) TCP, and the injectability rate for 50% (wt/polymer wt) HA-TSBC was slightly slower than that of 25% (wt/polymer wt) HA. When extruding the cement, no separation of phases (filter-pressing) occurred. Further, there was no clogging or clumping inside the mixing nozzle.

Although the 50% (wt/polymer wt) Brushite-TSBC composition resulted in an increased extrusion time as compared with the control composition, analysis of the polymerized surface of these compositions illustrates that the particles are still able to fully incorporate throughout the cement matrix. No particle clumping or agglomeration is visible in any of the cement matrices as illustrated in FIG. 9. The topography of the cement surfaces is very similar in each composition other than that of the control TSBC. The goal of incorporating CaP additives into the cement matrix is to provide bioactivity to the cement. It is worth noting, that after the addition of each calcium phosphate additive to the TSBC, the topography became more similar to cancellous bone. This is a beneficial aspect because unlike the surface of the control TSBC, the additive compositions provide improved venues for osteoconductive and osteointegrative surfaces for bone growth and reformation.

Other parameters must be still evaluated in future studies. The material shelf-life (both with and without fillers), for example, is essential to investigate because the influence of age could potentially affect a number of different characteristics of the cement. It was also noticed that the volume of cement left in the cartridge influenced the injectability time. This is likely due to the increase in pressure built up within the cartridge as the volume decreases, and has prompted a redesign of the entire cartridge system to minimize pressure within the system. Because of the small sample size necessary to execute the gel point analysis of the cement mixtures, the setting time associated with these experiments is falsely low. The setting time and exothermal temperature of the cement is sample size dependent, and thus further studies need to be conducted using ASTM standard F451 (ASTM Standard F451, 2008) to investigate these properties. Additionally, because the CaP fillers at 50% (wt/polymer wt) did not detrimentally influence the rheological characteristics of the TSBC cements, a future study investigating the effects of CaP additives in TSBC systems at higher concentrations (>50%) needs to be conducted.

In conclusion, this study was able to establish that the incorporation of calcium phosphate additives at concentrations of 25% and 50% (wt/polymer wt) into a two-solution bone cement system does not significantly result in changes to its cement viscosity, nor alter the pseudoplasticity or shear-thinning characteristics of the material. The increased swelling time of the TSBCs as compared to powder-liquid formulations allows for full incorporation of the calcium phosphate phase into the polymer matrix. This incorporation resulted in improved particle dispersion throughout the cement and prevented clumping or filter-pressing during cement extrusion. Furthermore, this high-viscosity, pseudoplastic, and osteoconductive TSBC system may be an effective alternative to conventional CaP-containing cements. The investigated cements are expected to find multiple applications in orthopedic surgery, providing improved interdigitation and integration with cancellous bone, which in turn will result in enhanced bone augmentation.

All of the compositions and/or methods disclosed and claimed herein can be made and executed without undue experimentation in light of the present disclosure. While the compositions and methods have been described in terms of specific embodiments, it will be apparent to those of skill in the art that variations can be applied to the compositions and/or methods and in the steps or in the sequence of steps of the method described herein without departing from the concept, spirit and scope of the disclosure. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept as defined by the appended claims.

### VII. REFERENCES

The following references, to the extent that they provide exemplary procedural or other details supplementary to those set forth herein, are specifically incorporated herein by reference.
U.S. Pat. No. 5,902,839
U.S. Pat. No. 8,383,734
Alt et al., Antimicrobial Agents and Chemotherapy, 48(11) 4084-4088, 2004.
ASTM Standard F451, 2008, “Standard Specifications for Acrylic Bone Cement,” ASTM International, West Conshohocken, Pa., 2008, DOI: 10.1520/FO451-08, www.astm.org.
Baroud et al., Acta Biomaterialia 1:357-363, 2005.
Baroud et al., Spine, 31(22):2562-2568, 2006.
Bechert et al., Nat. Med. 6:1053-1056, 2000.
Blattert et al., Spine, 34:108-114, 2009.
Bohner & Baroud, Biomaterials, 26(13): 1553-63, 2005.
Botelho, SADJ, 59(2) 51-53, 2004.
Chen et al., Chang Gung Med 1, 34:352-359, 2011.
Choi et al., J. Biomechanics 23(11), 1103-1113. 1990.
Grover et al., Biomaterials 24, 4133-4141, 2003.
Guvenm & Uzun, J. Hosp Infect. 53: 91-6, 2003.
Hasenwinkel et al., J. Biomed. Mater. Res. 47(1):36-45, 1999.
Hasenwinkel et al., J. Biomed Mater Res 59(3):411-421, 2002.
Hendriks et al., Biomaterials, 25: 545-556, 2004.
Holt, et al., J. Endodontics 33(7), 844-847, 2007.
Ishiguro et al., J. Orthop Surg., 18:346-351, 2010.
Jones & Hench, Current Opinion Solid State Materials Sci., 7(4-5) 301-307, 2003.
Josefsson et al., Clin. Othop., 159: 194-200, 1981.
Klekamp et al., J. Arthroplasty, 14(3): 339-346, 1999.
Lacy et al., Clin Infect Dis., 27: 23-7, 1998.
Lautenschlager et al., J. Biomed. Mater. Res., 10(6): 929-938, 1976.
Low et al., J. Biomed Mater Res B Appl Biomater, 94B:273-286, 2010.
Powles et al., J. Bone Jt. Surg. Br., 80: 607-10, 1988.
Qingsong & Salovey, Polymer Eng Sci, 44(3):452-462, 2004.
Rodrigues et al., J. Biomed Mater Res B Appl Biomater, 91(1):248-256, 2009.
Rosenthal et al., Inv. Med. Res., 4(5): 296-304, 1976.
Ryu, et al., World Neurosurg., 73:408-411, 2010.
Shahi et al., JOE, 33(12): 1429-32, 2007.
Tamimi et al., Acta Biomaterialia 8, 474-487, 2012.
Trippel, J. Bone Jt Surg Am., 68: 1297-302, 1986.
U.S. Patent Publication 2009/0239970
Van de Belt et al., Nat. Med., 5: 358-9, 1999.
Weiss et al., J. Long-Term Effects of Medical Implants, 13(1):41-47, 2003.
Wilke et al., Spine, 31:2934-2941, 2006.
Wroblewski et al., Acta Orthop. Scand., 57: 413-4, 1986.
Yang et al., J Biomed Mater Res B Appl Biomater, 100:1911-1921, 2012.

1. An orthopedic bone cement comprising:
(a) a particles;
(b) a linear poly(methyl methacrylate) polymer;
(c) a methyl methacrylate monomer;
(d) a bioactive calcium phosphate-containing agent;
(e) a porosity agent;
(f) an initiator of polymerization; and
(g) an activator of the initiator,
(h) an inhibitor of polymerization.

2. The orthopedic bone cement of claim 1, wherein the total polymer to monomer ratio is between about 1:1 and 2:1.

3. The orthopedic bone cement of claim 1, wherein the total polymer to monomer ratio is between about 1:1 and 1.5:1.

4. The orthopedic bone cement of claim 1, wherein the total polymer to monomer ratio is between about 1:2 and 1:3.

5. The orthopedic bone cement of claim 1, wherein said poly(methyl methacrylate) has a molecular weight of about 60,000 to about 100,000 daltons.

6. The orthopedic bone cement of claim 1, wherein said bioactive calcium phosphate-containing agent is tricalcium phosphate, brushite or hydroxyapatite.

7. The orthopedic bone cement of claim 1, wherein said particles may be microspheres.

8. The orthopedic bone cement of claim 1, wherein said particles are between about 0.1 and 400 micrometers.

9. The orthopedic bone cement of claim 7, wherein said particles are between about 1.0 and about 100 micrometers.

10. The orthopedic bone cement of claim 1, wherein the powder-to-liquid ratio is about 1:1 to about 2:1.

11. The orthopedic bone cement of claim 10, wherein the powder-to-liquid ratio is about 1:1 to about 1.5:1.

12. The orthopedic bone cement of claim 1, wherein said cement further comprises an antimicrobial agent.

13. The orthopedic bone cement of claim 1, wherein said antimicrobial agent is an antibiotic or an antifungal agent.

14. The orthopedic bone cement of claim 13, wherein said antibiotic is chlorhexidine, vancomycin, tobramycin or gentamicin.

15. The orthopedic bone cement of claim 1, wherein said antimicrobial agent is present at 1-10 weight percent, including 1-8 weight percent and 1-6 weight percent.

16. The orthopedic bone cement of claim 1, wherein the bioactive calcium phosphate-containing agent is included at 25-50% wt/wt of PMMA.

17. The orthopedic bone cement of claim 1, wherein the bioactive calcium phosphate-containing agent is included at 25% wt/wt of PMMA.

18. The orthopedic bone cement of claim 1, wherein the bioactive calcium phosphate-containing agent is included at 30% wt/wt of PMMA.

19. The orthopedic bone cement of claim 1, wherein the bioactive calcium phosphate-containing agent is included at 40% wt/wt of PMMA.

20. The orthopedic bone cement of claim 1, wherein the bioactive calcium phosphate-containing agent is included at 50% wt/wt of PMMA.

21. The orthopedic bone cement of claim 1, wherein said monomer comprises of a mixture of methyl methacrylate monomer (MMA) and styrene.

22. The orthopedic bone cement of claim 1, wherein said porosity agent is lactose.

23. The orthopedic bone cement of claim 1, wherein said initiator of polymerization is benzoyl peroxide.

24. The orthopedic bone cement of claim 1, wherein said activator of the initiator is electromagnetic radiation with wavelength between 10⁻⁷ to 10⁻¹⁰ meters.

25. The orthopedic bone cement of claim 1, wherein said inhibitor of polymerization is hydroquinone.

26. The orthopedic bone cement of claim 1, wherein said activator of the initiator is N,N-Dimethyl-Para-Toluidine.

27. A method of treating a subject for a bone-related disorder comprising administering to a site of bone damage, loss or deficiency a bone cement according to claim 1.

28. The method of claim 27, further comprising implanting into said subject, medical device, or appliance.

29. The method of claim 27, wherein said treating comprises an orthopedic, periodontal, neurosurgical, oral or maxillofacial procedure.

30. The method of claim 27, wherein said treating comprises repair of a simple fracture, compound fracture or non-union; external or internal fixation; joint reconstruction, arthrodesis, arthroplasty or cup arthroplasty of the hip; femoral or humeral head replacement; femoral head surface replacement or total joint replacement; repair of the vertebral column, spinal fusion or internal vertebral fixation; tumor surgery; deficit filling; discectomy; laminectomy; excision of spinal cord tumors; an anterior cervical or thoracic operation; repair of a spinal injury; treatment of scoliosis, treatment of lordosis; kyphosis treatment; intermaxillary fixation of a fracture; mentoplasty; temporomandibular joint replacement; alveolar ridge augmentation or reconstruction; as part of an inlay osteoimplant; implant placement and revision; sinus lift; a cosmetic procedure; or the repair or replacement of the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal bones and/or metatarsal bones.
